(19) United States
(12) Patent Application Publication
(10) Pub. No.: US 2012/0021937 A1
(43) Pub. Date: Jan. 26, 2012

(54) DETECTION OF CARCINOMA IN SITU IN SEMEN SPECIMENS

(75) Inventors: Kristian Almstrup, Roskilde (DK); Ewa Rajpert-De Meyts, Greve (DK); David Mobjerg Kristensen, Valby (DK); Niels Erik Skakkebaek, Farum (DK); Si Brask Sonne, Bronshoj (DK); John Erik Nielsen, Copenhagen S (DK)

(73) Assignees: Kobenhavns Universitet, Copenhagen K (DK); Rigshospitalet, Copenhagen (DK)

(21) Appl. No.: 13/124,405

(22) PCT Filed: Oct. 8, 2009
(86) PCT No.: PCT/DK2009/050269

§ 371 Date: Oct. 5, 2011

(30) Foreign Application Priority Data
Oct. 16, 2008 (DK) PA 2008 01448

(51) Int. Cl. C40B 30/04; C12Q 1/42; G01N 33/577; G01N 33/574; C12Q 1/68

(52) U.S. Cl. 506/9; 435/7.23; 435/6.12; 435/6.11; 435/7.1; 435/21; 435/7.92

(57) ABSTRACT

The present invention provides a method for the detection of testicular cancer and/or precursors hereof by screening a sample for the presence of at least two markers in the same cell, wherein the sample is a semen sample and/or an ejaculate from a male human being.

All patent and non-patent references cited in the application, or in the present application, are also hereby incorporated by reference in their entirety.

### FIELD OF INVENTION

The present invention relates to a method for the detection of carcinoma in situ (CIS) testis and/or related conditions such as testicular cancer by identification of at least two markers in an ejaculated semen sample from a male human being.

### BACKGROUND OF INVENTION

Testicular germ cell tumor, aka testicular cancer, has over the past several decades had an increasing incidence, making it the most common cancer in young males today. The disease typically presents in young males (aged 15-45 years) and most typically while the men are in their mid twenties. The lifetime risk of acquiring a testicular germ cell tumor (TGCT) is 0.5 to 1% for all males.

Testicular carcinoma in situ (CIS) is the common precursor of nearly all testicular germ cell tumors (TGCTs) that occur in young adults (Skakkebaek, 1972). Epidemiological evidence and evidence based on immunohistochemical analysis of germ cells indicate that CIS originates early in life, most probably from gonocytes that failed to differentiate to mature spermatogonia (Rajpert-De Meyts 2006). CIS cells then progress to an overt TGCT after puberty. The precise nature of the molecular events underlying the initiation of transformation from the gonocyte to the CIS cell has not yet been elucidated, and the following progression into overt tumors remains largely unknown. It is however known that the presence of CIS cells almost invariably will give rise to a tumor at some stage later in life.

Testicular cancer has one of the highest cure rates of all cancers: in excess of 90%; and close to 100%, if it has not widely metastasized or transformed to the forms of teratomas that are refractory to treatment. Even for the relatively few cases in which malignant cancer has spread widely, chemotherapy offers a cure rate of at least 85% today. However, a good outcome for testicular cancer as for any cancer is dependent on an early diagnosis. Furthermore, although testicular cancer patients may indeed be likely to survive the diagnosis, the disease often has major additional implications including an increased risk of fertility problems (often complete sterility), a need for testosterone replacement therapy, besides an increased risk of developing other cancers and cardiovascular diseases.

The cardinal diagnostic finding in the patient with testis cancer is a palpable mass in the testis, with or without enlargement or pain in the adolescent or young adult male. An ultrasound examination of the testis is necessary to assess the mass in the preliminary manner. If it does resemble a solid tumor, the treatment of choice is orchiectomy, with an intra-operation evaluation of a frozen tissue sample to exclude a possibility of a rare benign mass, such as a Leydig cell adenoma, which may be treated with the testis-preserving excision of the mass. In all cases of germ cell tumors, the entire testis along with attached structures such as the epididymis and spermatic cord must be removed. Partial excision is an incorrect procedure as in nearly all cases pre-invasive CIS cells are present in the supposedly normal tissue adjacent to the tumor. At the same time, a biopsy of the contra-lateral testis is often taken to exclude a possibility of the bilateral disease. Based on the ensuing evaluation of the tumor type and the presence of tumor spread, additional treatment may include chemotherapy, or radiation.

Obviously, the presence of a mass, which is the tumor itself, is a late stage at which to diagnose testicular cancer, which can be diagnosed earlier at the preinvasive stage of CIS. However, for two reasons this seldom happens: firstly, CIS is usually asymptomatic; secondly, the diagnosis of CIS can only be given following a surgical biopsy and this diagnostic procedure is generally only carried out in patients at-risk (e.g. with a history of cryptorchidism) or after clinical examination with suspicious outcome (atrophic testis) and ultrasonography (microlithiasis or very irregular echo pattern) (Roth et al., 2000). In such cases, the only diagnostic procedure currently available is a surgical biopsy, most often performed as an open incision of a tissue sample and sometimes as a needle-biopsy. After proper histological processing the sample has to be examined by a pathologist. The finding of CIS or local microinvasive spread of tumor cells will be in most cases treated by orchiectomy, with the exception of bilateral testicular cancer (or if the patient has only one testis), which is treated by irradiation, sometimes with adjuvant chemotherapy.

Due to the invasiveness and the high cost of the current diagnostic methods, as well as the obvious negative impact of the treatment of the later stages of the disease, great efforts have been invested in the development of methods for detecting CIS in semen samples. Semen from patients with testicular cancer contains cells with abnormal morphology, but the morphology alone is not sufficient to detect CIS without certainty, because the morphology of seminal cells other than sperm cells is poorly preserved (Czarplicki et al., 1987). The use of biochemical markers for detection of CIS is thus an area of great interest, and attempts have been made to find suitable markers: The antibody against M2A was found to detect CIS cells in semen by immunocytochemistry (Giwercman et al., 1988b; Meng et al., 1996), and the aneuploid DNA content and occasional presence of the isochomosome i(12p) was used by chromosomal in situ hybridization (Giwercman et al., 1990; Meng et al., 1998). However, both of these methods proved time-consuming and not sufficiently reliable to be used for diagnostic purposes in a clinical setting. Both false positive and false negative results were obtained due to partial degradation of cells and cell-surface antigens in the semen.

Promising results were obtained with antibodies against AP-2γ (AP-2gamma) (TFAP2C) on semen samples (Hoei-Hansen et al., 2005). Patients with TGCT and other cancers typically deliver several semen samples for cryopreservation prior to treatment, as the surgical, low-dose irradiation or chemotherapy treatment results in sterility. Surplus material from the cryopreservation was used in combination with control material from military conscripts and surplus of material from sub-fertile patients. Within this material a sub-fertile man with oligozoospermia was diagnosed successfully by immunocytochemical AP-2γ detection of CIS cells in semen (Hoei-Hansen et al., 2005a). In this initial investigation, no positive staining was found among the healthy controls and patients with other cancers (Hoei-Hansen et al., 2005). Hoei-Hansen et al (2007) confirmed and detailed their initial results with AP-2γ on a large group of patients and controls. In that study similar results were obtained with OCT3/4, whereas NANOG (a transcription factor) and PLAP (Placental alkaline phosphatase) were found to be unsuitable for CIS detection by immunocytochemical staining in semen samples due to considerable non-specific reactions. False positive results could not be ruled out and the conclusion was that the analysis performed does not meet the criteria for valid and sensitive diagnostic analysis.

There is thus an unmet need for a method for early, non-invasive detection of CIS having high specificity and accuracy.

### SUMMARY OF INVENTION

The present invention provides a method for early, non-invasive detection of CIS with high specificity and accuracy. A major advantage of the method of the present invention compared to the methods currently in use/disclosed in the prior art for the detection of testicular cancer is the non-invasiveness of the herein disclosed method. The present method relies on the examination of ejaculate/semen samples and does thus not require a biopsy or orchiectomy. The analysis of the ejaculate/semen sample is performed in a specific manner reducing the risk of obtaining false positive and false negative results.

The invention is based on the finding that the use of multiple markers makes it possible to screen ejaculate/semen samples for the presence of CIS, TGCT and/or derived cancers such as testicular carcinoma.

The main aspect of the present invention relates to a method for the detection of testicular cancer and/or precursors hereof by screening a sample for the presence of at least two markers in the same cell, wherein the sample is a semen sample and/or an ejaculate from a male human being.

Markers proven useful in semen diagnosis of testicular CIS/cancer are localized to the nucleus but are, as shown here, also expressed in a subset of cells of the epithelia of the urogential tract. As semen/ejaculate passes through the urogential system (i.e. vas deferens, epididymis, and the urethra), epithelial cells exfoliate to the ejaculate; for this reason, these markers have previously been found to cause false positive results. The present invention discloses a new method whereby nuclear markers with good CIS detection ability in ejaculate/semen samples are used in combination with other, non-nuclear markers, which are not expressed in the epithelia of the urogenital tract. The use of non-nuclear markers in ejaculate/semen samples is new, as cells in ejaculate/semen samples are up to three weeks old, and therefore are of less than optimal quality for the detection of non-nuclear markers. Over the course of several weeks the non-spermatozoa cells are embedded in the fluid that eventually becomes the ejaculate/semen sample, where the cells are exposed to proteases and other factors that severely damage their morphology. In fact, the plasma membrane is often compromised, and the cells thus leak cytoplasm and other cellular constituents. It is thus highly surprising, that a method has been developed that overcomes these problems and thus provides the solution to a long-standing need for a non-invasive screening method for the detection of testicular cancer and/or the precursors hereof such as testicular CIS cells.

Thus the key aspect of the present invention regards the use of a combination of markers such as at least one nuclear and one non-nuclear marker for the detection of cancerous/CIS cells in an ejaculate/semen sample.

A further embodiment regards the use of immunostaining and/or enzymatic assays for the detection of the at least one nuclear and one non-nuclear marker during the screening for cancerous/CIS cells in an ejaculate/semen sample

Furthermore an embodiment of the present invention regards the use of total ejaculate/semen samples. Preferably, the total ejaculate samples are treated in a manner making it possible to process the entire sample in one procedure.

### BRIEF DESCRIPTION OF DRAWINGS

FIG. 1 The human male reproductive system.

FIG. 2 Testicular dysgenesis syndromes (TDS), its causes and outcomes.

FIG. 3 Immunohistochemical and enzyme activity survey of expression of CIS markers and activity in epithelia from the urogenital tract.

FIG. 4 RT-PCR of selected CIS markers in urogenital tissues.

FIG. 5 Staining of CIS cells in semen samples using both alkaline phosphatase activity and AP-2γ immunohistochemistry.

FIG. 6 Representative cells found in ejaculates from an infertile male later found to harbour CIS cells in a testicular biopsy.

FIG. 7 Immunohistochemical staining for PLAP in a testicular biopsy taken from an infertile male with 4 out of 4 positive semen cytospins. A) Large overview. B) Higher magnification.

FIG. 8 Immunostaining of TCam-2 control cells demonstrating improved staining intensity when using a combination of antibodies

### DEFINITIONS

Allele: A member of a pair or series of different forms of a gene. Usually alleles are coding sequences, but sometimes the term is used to refer to a non-coding sequence. Alleles are known or recognized differences in DNA, RNA or protein sequences.
CIS: Abbreviation for ‘carcinoma in situ’.
Cryptorchidism: The absence of one or both testes from the scrotum.
Cytoplasm: The entire contents of a eukaryotic cell excluding the nucleus, and bounded by the plasma membrane. Used interchangeably with ‘cytosol’, which is the liquid phase of the cytoplasm.
Cytospin: A collected sample is fixed onto a microscope slide by centrifugation.
Detection: Herein the observation of a cancerous cell i.e. a testicular cancer/CIS cell by the methods herein disclosed.
Ejaculate: The fluid emitted from a male penis that contains, among other things, sperm. It may or may not contain sperm cells (spermatozoa) and herein used interchangeably with the expression ‘semen sample’.
False negative: Also known as ‘Type II error’, ‘error of the second kind’, ‘β error’; the error of failing to reject a null hypothesis when the alternative hypothesis is the true state of nature. In other words, this is the error of failing to observe a difference when in truth there is one.
False positive: Also known as ‘Type I error’, ‘error of the first kind, ‘α error’; the error of rejecting a null hypothesis when it is actually true. Plainly speaking, it occurs when a difference is observed when in truth there is none. A false positive normally means that a test claims something to be positive, when that is not the case.
Immunocytochemistry: The process of localizing proteins in cells by employing antibodies specific for the proteins of interest and using a method that allows the antibodies to bind to the proteins allowing visualization of possible sub-cellular localization.
Immunohistochemistry: The process of localizing proteins in cells of a tissue section by employing antibodies specific for the proteins of interest and using a method that allows the antibodies to bind to the proteins while in a relatively native setting in a biological tissue section.
Marker: An indicator signaling an event or condition in a biological system or sample giving a measure of status, exposure, susceptibility and more of the biological system, dependent on the marker. A marker is herein the presence of a gene or product(s) hereof, the presence or relative level of which alone or in combination with other markers may indicate a neoplastic and/or cancerous state.
Mutant: A term applied to a gene or product thereof, which differs from what is considered the “wild type” or native form of same, the difference most typically arising due to mutation.
Non-nuclear: As relating to any part of a eukaryotic cell, which is not the nucleus. Herein predominantly used as a description of a localization/expression pattern for various markers. Commonly herein non-nuclear localization is understood as detectable in a cytoplasmic localization.
Nuclear: As relating to the nucleus of a eukaryotic cell. Herein predominantly used as a description of a localization/expression pattern for various markers.
Precursor: Herein precursors are cells of a stage during cancerous development that is prior to an actual tumor formation, e.g. neoplastic and carcinoma in situ (CIS) cells.
Screening: The examination of a sample to pick up the few aberrant/cancerous cells that it may comprise. A screening may be performed manually or be an automated process.
Semen sample: Semen is generally the same fluid as released during an ejaculation;

herein a semen sample includes any sample comprising e.g. spermatozoa and which may be taken by e.g. expiration directly from a testicle or elsewhere in the male urogential system. May be used interchangeably with the term ‘ejaculate’.
Slide: A slide according to the present invention is meant to comprise microscope slides; standardized microscope slides are in the form of a thin sheet of glass used to hold objects (such as cells or tissues) for examination under a microscope.
Testicular cancer: Malignant tumor or cell from a testicle.
TGCT: Abbreviation for ‘testicular germ cell tumor’
PLAP: Placenta-like alkaline phosphatase, is used herein interchangeably with ‘alkaline phosphatase’ and ALPPL2
AP-2γ: AP-2gamma, AP2γ, used herein interchangeably with TFAP2C
OCT3/4: Used interchangeably with POU5F1

The present invention regards the use of at least two markers for the detection of conditions such as testicular carcinoma in situ (CIS), and/or testicular cancer and/or precursors hereof, in a semen or ejaculate sample. In a preferred embodiment, a nuclear and a cytoplasmic marker are used simultaneously.

### DETAILED DESCRIPTION OF THE INVENTION

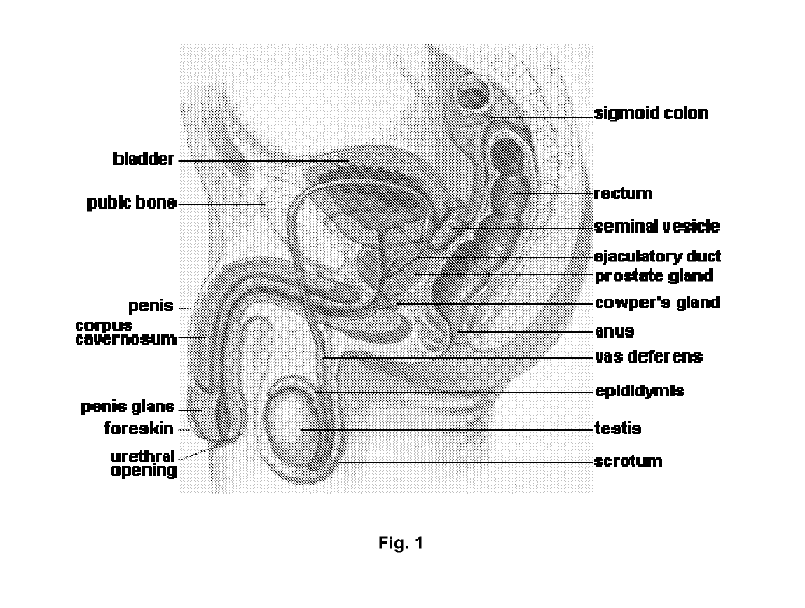

The human male reproductive system is a series of organs located outside of the body and around the pelvic region of a male that contribute towards the reproductive process. During ejaculation, sperm (or spermatozoa) leaves the penis in a fluid called seminal fluid. This fluid is produced by 3 types of glands; the seminal vesicle, the prostate gland, and Cowper's glands (bulbourethral glands). The male reproductive system is outlined in FIG. 1.

Testicular Dysgenesis Syndrome

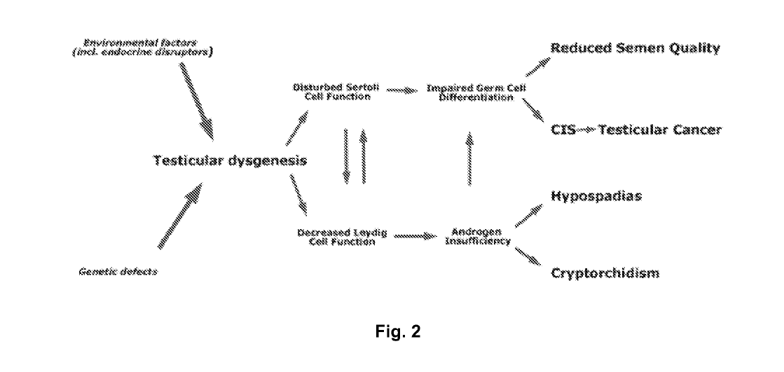

It has been proposed that a collection of adverse conditions in male reproductive health have their basis in a common origin; specific errors during the fetal development of testes. This collection of disorders is recognized as testicular dysgenesis syndrome (TDS), and it is likely to be caused by environmental factors in many cases, and by rare genetic disorders in others. Both primarily lead to variation in fetal hormonal levels, which again leads to the observed diseases and dysfunctions (FIG. 2) (Skakkebaek et al., 2001). FIG. 2 identifies the two sources of TDS, environmental factors and genetic defects, and their consequences. One pathway of impact, via disruption in Sertoli cell function, leads to reduced semen quality and testicular cancer. The other, through impacts on Leydig cell function, causes hypospadias and cryptorchidism. The common origin, TDS, thus leads to a cluster of related health effects.

At least four health effects may be attributed to TDS: testicular cancer, undescended testis, hypospadias, and lowered sperm quality. All four conditions tend to co-occur in the affected males and the following patterns have been observed:
Some cases of testicular cancer are caused by rare gene mutations. When they occur, they are often in combination with undescended testis and hypospadias.
Men with testicular cancer are more likely than normal to have experienced cryptorchidism.
Men with cryptorchidism are more likely than normal men to come to infertility clinics; the undescended testis is often manifests problems related to misdirected development, including impaired (or arrested) sperm production.
The contralateral non-cancerous testis of men with testicular cancer often has a series of malformations related to TDS.
Men with testicular cancer of one testis have extremely low sperm counts, much lower than what would be expected on the basis of the loss of one functional testis.
Research on sperm count of men who later developed testicular cancer confirms the presence of abnormal semen characteristics, including low sperm count, prior to the development of testicular cancer.
Men who later develop testicular cancer are likely to have had fewer children than normal men, an indication of reduced fertility, and also to have sired fewer male children.

The diseases and disorders related to TDS and the present invention are reviewed in the below.

The present invention relates to the detection of carcinoma in situ (CIS) cells originating in the testis by a non-invasive method. It is an aspect of the present invention that the method may be employed on ejaculate/semen samples from all males. The method is of special relevance for males diagnosed with one or more disorders or diseases that are characteristic of testicular dysgenesis syndrome.

CIS and Testicular Cancer

Carcinoma in situ (CIS) or intratubular germ cell neoplasms is an early form of carcinoma defined by the absence of invasion of surrounding tissues. In other words, the neoplastic cells proliferate in their normal habitat, hence the name ‘in situ’ (Latin for ‘in its place’). CIS cells of the testis may transform into either a seminoma or a non-seminoma. The seminoma retains a germ-cell-like phenotype, whereas the tumor known as non-seminoma or teratoma retains embryonic stem cell features; pluripotency and the ability to differentiate into virtually all somatic tissues. Non-seminomas comprise embryonal carcinoma (EC), various mixtures of differentiated teratomatous tissue components and extra-embryonic tissues, such as yolk sac tumor and choriocarcinoma. In addition to CIS, gonadoblastoma; a CIS-like lesion, occurs in dysgenetic testes and intersex gonads, which frequently may contain some ovarian structures. The only difference between CIS and gonadoblastoma concerns the overall architecture of the lesion in the surrounding gonad (CIS is found usually in single rows along the basement membrane and there is a single layer of Sertoli cells between CIS cells and the lumen of seminiferous tubules, while gonadoblastoma contains nests (clumps) of CIS-like cells surrounded by small somatic granulosa-like cells and sometimes spermatogonia-like cells). The morphology and gene-expression patterns of CIS cells and gonadoblastoma cells are indistinguishable, therefore, further in this application, the term CIS is used for both precursor lesions. Furthermore, the term CIS is herein used for the detection of testicular CIS.

Testicular cancers and their origin may be classified as follows:

Germ Cell Tumors of the Testis (TGCT)
40% mixed (usually teratoma plus another)
35% seminoma (germinoma of the testis)
20% embryonal carcinoma
5% teratoma (pure)
<1% yolk sac tumor
<1% choriocarcinoma
Gonadoblastoma (in men with disorders of sex differentiation)
<1% spermatocytic seminoma (in older men, not associated with CIS, usually benign)

Non-Germ Cell Tumors of the Testis
Sertoli-cell tumor (usually in children)
Leydig cell tumor (usually benign)
Rhabdomyosdarcoma or leiomyosarcoma

Secondary Tumors of the Testis
Lymphoma
Leukemic infiltration of the testis
Metastatic tumors

In broad terms, testicular cancer progresses through the following stages:
Stage I: the cancer remains localized to the testis.
Stage II: the cancer involves the testis and metastasis to retroperitoneal and/or Para-aortic lymph nodes (lymph nodes below the diaphragm).
Stage III: the cancer involves the testis and metastasis beyond the retroperitoneal and para-aortic lymph nodes. Stage III is further subdivided into non bulky stage III and bulky stage III.

It is an object of the present invention to provide a non-invasive method of detecting CIS, neoplastic cells of the testis and/or testicular cancer. The non-invasiveness of the method allows for easier screening, which allows earlier detection of the various cancerous stages, preferably CIS. Any type of cancerous cell may be detected by the method herein provided. However, the at least one cancerous cell preferably originates from the testis. The term “cancerous cell” will henceforth be used to cover any type of transformed i.e. neoplastic, CIS, benign or malignant cancer cell. The cancerous cell may be from a cancer at any stage in development as summarized in the above and may thus for example be a neoplastic cell, a CIS cell, and or a metastatic cancer cell. The cancerous cell may originally be a cell of any type of tissue, preferably an epithelial tissue as corresponds with the definition of carcinomas (as originating from epithelial cells). The cancerous cell may thus be of any type listed above, such as but not limited to: Germ cell tumors of the testis (TGCT), such as a teratoma, seminoma, embryonal carcinoma, pure teratoma, choriocarcinoma, gonadoblastoma, intratubular germ cell neoplasm's; Non-germ cell tumors of the testis, such as Sertoli cell or Leydig cell tumors; and Secondary tumors of the testis such as Lymphoma, Leukemic infiltration of the testis, and Metastatic tumors.

Preferably, the method of the present invention detects germ cell tumors of the testis (TGCT) and most preferably detects these at an early stage such as the CIS stage. Thus it is an object of the present invention to detect germ cell tumors of the testis and preferably to detect carcinoma in situ cells (CIS cells) originating from the testis.

The method is preferably employed on ejaculate/semen samples from mammalian males.

Thus an aspect of the present invention regards the detection of carcinoma in situ (CIS) by the method herein disclosed. Also, an aspect regards the detection of a secondary tumor of the testis, such as a lymphoma, leukemic infiltration of the testis or a metastatic tumor. Likewise another aspect regards the detection of a mixed tumor, a seminoma, an embryonal carcinoma, a teratoma, a choriocarcinoma or intratubular germ cell neoplasms (CIS). A further aspect regards the detection of non-germ cell tumors of the testis such as a Sertoli-Leydig cell tumor.

Cryptorchidism

Cryptorchidism is the absence of one or both testes from the scrotum. This usually represents failure of the testis to move, or “descend,” during fetal development from an abdominal position, through the inguinal canal, into the ipsilateral scrotum. About 3% of full-term and 30% of premature infant boys are born with at least one undescended testis, making cryptorchidism the most common birth defect of male genitalia. However, most testes descend by the first year of life (the majority within three months), making the true incidence of cryptorchidism around 1% overall.

Cryptorchidism is one of the health effects falling under the diagnosis of testicular dysgenesis syndrome. It is thus an aspect of the present invention to provide a method for the detection of cancerous cells such as CIS cells in individuals whom at any point in time have been diagnosed with cryptorchidism.

Hypospadias

Hypospadias is a birth defect of the urethra in the male that involves an abnormally placed urinary meatus (opening). Instead of opening at the tip of the glans of the penis, a hypospadic urethra opens anywhere along a line (the urethral groove) running from the tip along the underside (ventral aspect) of the shaft to the junction of the penis and scrotum or perineum. The urethral meatus opens on the glans penis in about 50-75% of cases; these are categorized as first degree hypospadias. Second degree (when the urethra opens on the shaft), and third degree (when the urethra opens on the perineum) occur in up to 20 and 30% of cases respectively. The more severe degrees are more likely to be associated with chordee, in which the phallus is incompletely separated from the perineum or is still tethered downwards by connective tissue, or with undescended testes (cryptorchidism).

Hypospadias is another of the health effects falling under the diagnosis of testicular dysgenesis syndrome. It is thus an aspect of the present invention to provide a method for the detection of cancerous cells such as CIS cells in individuals whom at any point in time have been diagnosed with hypospadias.

Semen Quality—Low Sperm Count

Semen quality is a measure of the ability of semen to accomplish fertilization. Thus, it is a measure of fertility in a man. It is the sperm in the semen that are of importance, and therefore semen quality involves both sperm quantity and sperm quality. Low sperm count/low semen quality may be due to many different factors, some genetic, some environmental. Azoospermia, aspermia, oligospermia and oligozoospermia are all diagnoses related to low or no sperm and are thus aspects of the present invention. Primarily, any diagnosis of low or no fertility in a male is for several reasons cause for concern, and therefore it is an aspect of the present invention to provide a method for the detection of cancerous cells such as CIS cells in individuals whom at any point in time have been diagnosed with low sperm count, low or bad semen quality, low fertility or infertility. Furthermore, low semen quality is one of the health effects falling under the diagnosis of testicular dysgenesis syndrome making it an aspect of the present invention to provide a method for the detection of cancerous cells such as CIS cells in semen samples and/or ejaculates from males diagnosed with low or no fertility.

Markers

The present invention relates to the specific and sensitive detection of CIS cells in semen/ejaculate samples. This is a novel and surprising approach, as the use of a cytoplasmic or cell-membrane marker previously has been deemed unreliable due to the lack of accuracy and/or specificity in discerning CIS cells from other, non-spermatozoa cells in a semen sample. The CIS cells travel along with the spermatozoa and increasing amounts of seminal fluid through the urogential system from the ducts of the testicles, through the epididymis, and via vas deferens into the ejaculatory duct and finally into the urethra, which all are tissues lined with epithelial cells. Therefore the final ejaculate consistently comprises exfoliated cells derived from the lining of these structures, which are not CIS cells, but due to their gene expression patterns nevertheless express many of the markers known as CIS markers. For example, many of the well-known nuclear CIS markers including AP-2γ, OCT3/4 have been found in self-renewing basal epithelia and connecting tissues of the vas deferens, epididymis, seminal vesicle (Vesicula seminalis), and prostate. The presence of these markers has previously given rise to false positive staining in semen samples. On the contrary, a range of non-nuclear/cytosolic markers have with the present investigation (see Examples) been found not to be expressed in tissues connected to the vas deferens and thus be more selective markers for CIS. The present invention, using a combination of non-nuclear and nuclear markers of CIS, results in the specific detection of CIS cells and circumvention of false positive cells originating from the urogenital tract.

It is an object of the present invention to provide a method of detecting cancerous cells in semen and/or ejaculate samples from mammalian males. The method of detection relies on the detection of specific genes and/or their products, which may be used as markers for CIS. By genes and/or their products is meant the detection of the presence of the genes of interest and any alleles and/or mutants hereof as well as their transcriptional and possible subsequent translational products including, but not limited to pre-mRNA, hnRNA, mRNA, smRNA, any antisense or regulatory RNA such as but not limited to: miRNA, siRNA, piRNA, and further smRNA, snoRNA, tRNA, rRNA, protein, polypeptide, peptide, modified (such as post-translationally modified) protein, peptide and/or polypeptides or fragments of proteins and/or polypeptides encoded for by the genes of interest and/or their alleles and/or mutants.

An aspect of the present invention relates to the use of at least one marker for the detection of cancerous cells, preferably at least two markers for the detection of cancerous cells. Thus, any number of markers such as one, two, three, four, five, six, seven, eight, nine, ten or more markers may be used in combination for the detection of cancerous cells in a semen and/or ejaculate sample. Preferably one marker alone or two or three markers are used in combination for the detection of cancerous cells in a semen and/or ejaculate sample. Most preferably two markers are used in combination for the detection of cancerous cells in a semen and/or ejaculate sample.

A further aspect of the present invention regards the subcellular localization of the markers used for the detection of cancerous cells. Cells of mammals are typical eukaryotic cells comprising within the cell membrane the nucleus and the cytoplasm, which surrounds the nucleus. Within the cytoplasm, organelles and structures other than the nucleus may be found; these include: the endoplasmatic reticulum (rough and smooth), Golgi apparatus, mitochondria, vesicles, vacuole(s), centrioles, cytoskeleton, peroxisomes and lysosomes and within the nucleus: the nucleolus, and the chromosomes. Furthermore, ribosomes and other very large complexes may be identified by microscopy, staining and other methods known to persons skilled in the art. Preferably, the markers of the present invention localize to/can be detected in the nucleus, cytoplasm and/or mitochondria of the cancerous cell.

It is an object of the present invention to provide a method of detecting cancerous cells by using at least one and preferably two or more markers having distinct subcellular localizations. Thus, the marker may bind to, or be found within, on, or surrounding any of the abovementioned organelles and/or structures. The markers may co-localize i.e. they localize to the same subcellular compartment, or they may localize to different subcellular compartments. In a preferred embodiment, the at least two different markers localize to different subcellular compartments. Preferably, the at least two markers localize to the nucleus or not, thereby meaning e.g. the cytoplasm. Thus, preferably one marker localizes to the nucleus and the other marker localizes somewhere else. The non-nuclear localization is preferably cytoplasmic.

Genes of interest, and hence proteins and so forth of relevance to the present invention include, but are not limited to any genes and/or proteins that are differentially or exclusively expressed in connection with cancer, preferably cancer of the testis. By differentially expressed is meant levels of expression that in the cancerous cells are above or below the levels observed in cells, preferably of the same origin (e.g. tissue or cell type), that are not cancerous and/or transformed. Such genes and/or proteins include but are not limited to e.g. the 895 genes detected using cDNA microarray analysis, which are expressed at significantly greater levels in human embryonic stem (ES) cells and embryonic carcinoma cell lines than in control samples. These 895 genes are candidates for involvement in the maintenance of a pluripotent-undifferentiated phenotype, a phenomenon that may lead to neoplastic development and CIS. These genes are to be found in Spreger et al; “Gene expression patterns in human embryonic stem cells and human pluripotent germ cell tumors”, (PNAS, 100:13350-13355, 2003) and are hereby all incorporated by reference. Likewise genes/proteins identified as CIS markers in Almstrup et al. 2007 are hereby all incorporated by reference. Of particular relevance are the biomarkers identified in WO2005/103703 which were found to be markers of testicular carcinoma in situ and cancers derived there from (see table 1 of the application) The biomarkers were found useful in expression profiling (e.g. microarrays) and are all hereby incorporated by reference.

A list of preferred cytoplasmic and nuclear markers useful for detection according to the present invention by any of the procedures outlined in the below is given in Table 1 below and a list of most preferred marker genes/proteins is given in Table 2 below.

Preferred markers of CIS that localize to the nucleus include, but are not limited to: TFAP2C, POU5F1, NANOG, SOX2, SOX15, SOX17, E2F1, IFI16, TEAD4, TLE1, TATDN2, NFIB, LMO2, MECP2, HHEX, XBP1, RRS1, MYCN, ETV4, ETV5, MYCL1, HIST1H1C, WDHD1, RCC2, TP53, and MDC1. The most preferred nuclear markers for use in the method according to the present invention for the detection of CIS in a semen/ejaculate sample are: TFAP2C, POU5F1, NANOG and TP53. It is an aspect of the present invention that the at least one marker used for the detection of CIS/cancerous cells in a semen/ejaculate sample includes at least one of the markers of above. In another aspect, at least two markers of the abovementioned markers are used for the detection, by co-localization, of CIS cells in a semen sample.

Preferred markers of CIS that do not localize to the nucleus include, but are not limited to: ALPPL2, ALPL, DPPA4, TCL1A, CDH1, GLDC, TCL1A, DPPA4, CDK5, CD14, FGD1, NEURL, HLA-DOA, DYSF, MTHFD1, ENAH, ZDHHC9, NME1, SDCBP, SLC25A16, ATP6AP2, PODXL, PDK4, PCDH8, RAB15, EVI2B, LRP4, B4GALT4, CHST2, FCGR3A, CD53, CD38, PIGL, CKMT1B, RAB3B, NRCAM, KIT, ALK2, PDPN, HRASLS3, and TRA-1-60. The most preferred non-nuclear markers for use in the method according to the present invention for the detection of CIS in a semen/ejaculate sample are: ALPPL2, ALPL, KIT and PDPN. It is an aspect of the present invention that the at least one marker used for the detection of CIS/cancerous cells in a semen/ejaculate sample includes at least one of the markers of above. In another aspect, at least two markers of the abovementioned markers are used for the detection, by co-localization, of CIS cells in a semen sample.

TABLE I
Official gene names of genes/proteins which are preferred
CIS markers, their synonym(s) and subcellular localization

| | | |
|---|---|---|
| Gene | | |
| symbol | Synonym | Gene name |

Nucleus

| | | |
|---|---|---|
| TFAP2C | AP2γ, | transcription |
| | AP2-gamma, | factor AP-2 |
| | ERF1, | gamma |
| | TFAP2G, | |
| | hAP-2g | |
| POU5F1 | OCT3/4, | POU class 5 |
| | OCT3, | homeobox 1 |
| | OCT4, | |
| | MGC22487 | |
| NANOG | FLJ12581, | Nanog |
| | FLJ40451 | homeobox |
| SOX2 | — | SRY (sex |
| | | determining |
| | | region Y)-box 2 |
| SOX15 | SOX27, | SRY (sex |
| | SOX26 | determining |
| | | region Y)-box 15 |
| SOX17 | — | SRY (sex |
| | | determining |
| | | region Y)-box 17 |
| E2F1 | RBP3 | E2F transcription |
| | | factor 1 |
| IFI16 | IFNGIP1, | interferon, |
| | PYHIN2 | gamma-inducible |
| | | protein 16 |
| TEAD4 | TEF-3, | TEA domain |
| | TEFR-1, | family member 4 |
| | EFTR-2, | |
| | RTEF-1 | |
| TLE1 | ESG1, | transducin-like |
| | GRG1, | enhancer of split |
| | ESG | 1 (E(sp1) homolog, |
| | | Drosophila) |
| TATDN2 | KIAA0218 | TatD Dnase domain |
| | | containing 2 |
| NFIB | NFI-RED, | nuclear factor I/B |
| | NFIB2, | |
| | NFIB3 | |
| LMO2 | TTG2, | LIM domain only 2 |
| | RHOM2, | (rhombotin-like 1) |
| | RBTN2 | |
| MECP2 | RTT, | methyl CpG |
| | MRX16, | binding protein 2 |
| | MRX79 | (Rett syndrome) |
| HHEX | HEX, | hematopoietically |
| | HOX11L- | expressed homeobox |
| | PEN | |
| XBP1 | — | X-box binding |
| | | protein 1 |
| RRS1 | KIAA0112 | RRS1 ribosome bio- |
| | | genesis regulator |
| | | homolog (S. |
| | | cerevisiae) |
| MYCN | bHLHe37 | v-myc |
| | | myelocytomatosis |
| | | viral related |
| | | oncogene, |
| | | neuroblastoma |
| | | derived (avian) |
| ETV4 | E1A-F, E1AF | ets variant 4 |
| ETV5 | ERM | ets variant 5 |
| MYCL1 | LMYC, | v-myc |
| | bHLHe38 | myelocytomatosis |
| | | viral oncogene |
| | | homolog 1, lung |
| | | carcinoma |
| | | derived (avian) |
| HIST1H1C | H1.2 | histone cluster 1, |
| | | H1c |
| WDHD1 | AND-1 | WD repeat and |
| | | HMG-box DNA |
| | | binding protein 1 |
| RCC2 | TD-60 | regulator of |
| | | chromosome |
| | | condensation 2 |
| TP53 | p53, LFS1 | tumor protein |
| | | p53 |
| MDC1 | NFBD1, | mediator of DNA |
| | KIAA0170, | damage |
| | Em: AB023051.5 | checkpoint 1 |

Non-nucleus

| | | |
|---|---|---|
| ALPPL2 | PLAP | alkaline |
| | | phosphatase, |
| | | placental-like 2 |
| ALPL | TNSALP | alkaline |
| | | phosphatase, |
| | | liver/bone/kidney |
| DPPA4 | FLJ10713 | developmental |
| | | pluripotency |
| | | associated 4 |
| TCL1A | TCL1 | T-cell leukemia/ |
| | | lymphoma 1A |
| CDH1 | uvomorulin, | cadherin 1, type 1, |
| | CD324 | E-cadherin |
| | | (epithelial) |
| GLDC | GCSP, | glycine |
| | NKH | dehydrogenase |
| | | (decarboxylating) |
| CDK5 | PSSALRE | cyclin-dependent |
| | | kinase 5 |
| CD14 | | CD14 molecule |
| FGD1 | ZFYVE3 | FYVE, RhoGEF |
| | | and PH domain |
| | | containing 1 |
| NEURL | h-neu, | neuralized homolog |
| | RNF67, | (Drosophila) |
| | NEURL1 | |
| HLA-DOA | HLA-D0- | major histo- |
| | alpha | compatibility |
| | | complex, class II, |
| | | DO alpha |
| DYSF | FER1L1 | dysferlin, limb |
| | | girdle muscular |
| | | dystrophy 2B |
| | | (autosomal |
| | | recessive) |
| MTHFD1 | MTHFC, | methylenetetra- |
| | MTHFD | hydrofolate |
| | | dehydrogenase |
| | | (NADP+ depen- |
| | | dent) 1, |
| | | methenyltetra- |
| | | hydrofolate |
| | | cyclohydrolase, |
| | | formyltetrahydro- |
| | | folate synthetase |
| ENAH | FLJ10773, | enabled homolog |
| | NDPP1, | (Drosophila) |
| | MENA | |
| ZDHHC9 | ZNF379, | zinc finger, DHHC- |
| | CGI-89, | type containing 9 |
| | ZNF380 | |
| NME1 | NM23, | non-metastatic |
| | NM23-H1 | cells 1, protein |
| | | (NM23A) |
| | | expressed in |
| SDCBP | SYCL | syndecan binding |
| | | protein (syntenin) |
| SLC25A16 | GDA, | solute carrier family |
| | D10S105E, | 25 (mitochondrial |
| | HGT.1, | carrier; Graves |
| | ML7 | disease |
| | | autoantigen), |
| | | member 16 |
| ATP6AP2 | M8-9, | ATPase, H+ |
| | APT6M8-9, | transporting, |
| | ATP6M8-9 | lysosomal |
| | | accessory protein 2 |
| PODXL | PCLP, | podocalyxin-like |
| | Gp200, PC | |
| PDK4 | — | pyruvate |
| | | dehydrogenase |
| | | kinase, isozyme 4 |
| PCDH8 | PAPC, | protocadherin 8 |
| | ARCADLIN | |
| RAB15 | | RAB15, member |
| | | RAS onocogene |
| | | family |
| EVI2B | D17S376, | ecotropic viral |
| | EVDB | integration site 2B |
| LRP4 | MEGF7 | low density |
| | | lipoprotein |
| | | receptor-related |
| | | protein 4 |
| B4GALT4 | beta4Gal-T4 | UDP-Gal: betaGlcNAc |
| | | beta 1,4- |
| | | galactosyltransferase, |
| | | polypeptide 4 |
| CHST2 | C6ST | carbohydrate (N- |
| | | acetylglucosamine-6-O) |
| | | sulfotransferase 2 |
| FCGR3A | CD16, | Fc fragment of IgG, |
| | CD16a | low affinity IIIa, |
| | | receptor |
| CD53 | TSPAN25 | CD53 molecule |
| CD38 | — | CD38 molecule |
| PIGL | — | phosphatidylinositol |
| | | glycan anchor bio- |
| | | synthesis, class L |
| CKMT1B | UMTCK | creatine kinase, |
| | | mitochondrial 1B |
| RAB3B | | RAB3B, member |
| | | RAS oncogene |
| | | family |
| NRCAM | KIAA0343, | neuronal cell |
| | Bravo | adhesion molecule |
| KIT | CD117, | v-kit Hardy- |
| | SCFR, C- | Zuckerman 4 feline |
| | Kit | sarcoma viral |
| | | oncogene homolog |
| ACVR1 | SKR1, | activin A receptor, |
| | ALK2, | type I |
| | ACVR1A | |
| PDPN | T1A-2, | podoplanin |
| | Gp38, | |
| | aggrus, | |
| | GP40, | |
| | PA2.26 | |
| PLA2G16 | HRASLS3, | phospholipase A2, |
| | HREV107, | group XVI |
| | H-REV107-1, | |
| | HREV107-3, | |
| | MGC118754., | |
| | AdPLA | |
| TRA-1-60 | | |

TABLE II
Most preferred genes for use as CIS markers

| Nucleus | | Non-nucleus | |
|---|---|---|---|
| Gene | | Gene | |
| name | Synonym | name | Synonym |
| TFAP2C | AP2γ, AP2-gamma, | ALPPL2 | PLAP |
| | ERF1, TFAP2G, | | |
| | hAP-2g | | |
| POU5F1 | OCT3/4, OCT3, OCT4, | ALPL | TNSALP |
| | MGC22487 | | |
| NANOG | FLJ12581, FLJ40451 | KIT | CD117, SCFR, C-Kit |
| TP53 | p53, LFS1 | PDPN | T1A-2, Gp38, aggrus, |
| | | | GP40, PA2.26 |

As described above, an embodiment of the present invention relates to the use of two or more markers which localize to different compartments within the at least one cancerous cell/CIS cell. These compartments are preferably the nuclear and the non-nuclear compartment. Thus, of the markers herein included non-limiting, but preferred examples of combinations of markers localizing to different compartments include: the nuclear marker TFAP2C used in combination with at least one of the non-nuclear markers: ALPPL2, ALPL, KIT and/or PDPN; likewise POU5F1 (nuclear marker) used in combination with any of the non-nuclear markers: ALPPL2, ALPL, KIT and PDPN; similarly the nuclear marker NANOG used in combination with at least one/any of the non-nuclear markers: ALPPL2, ALPL, KIT and/or PDPN; and also TP53 (a nuclear marker) is preferably used in combination with any of the non-nuclear markers: ALPPL2, ALPL, KIT and/or PDPN. The reverse combinations are obviously also preferred e.g. using the non-nuclear marker ALPPL2 in combination with at least one/any of the nuclear markers: TFAP2C, POU5F1, NANOG and/or TP53. Most preferably, TFAP2C is used in combination with ALPPL2.

The advantage of using both a non-nuclear/cytosolic marker (such as e.g. ALPPL2) and a nuclear marker (such as e.g. TFAP2C) is a co-localization of signals originating from the cytosol and the nucleus. This greatly reduces the risk of false positive read-outs from analyzing the sample for the detection of CIS markers.

The nuclear CIS markers are retained from their in situ localization in the seminiferous tubules until ejaculated, while the cytoplasmic markers sometimes are degraded during this travel. The nuclear markers are however, as shown here, also expressed in the epithelia of the urogenital tract and sometimes also by infiltrating lymphocytes, which may yield false positive cells if based on a single nuclear staining. Moreover, analyses on single staining procedures always have to deal with artifacts giving positive staining.

However, using a combination of a robust nuclear marker together with a cytosolic marker significantly enhances the specificity of the assay.

Individuals to be Examined

The method of detection of cancerous cells, especially CIS cells in semen and/or ejaculate samples may be employed on semen/ejaculate samples from any mammalian male. Preferably, the method is practiced on ejaculated semen samples from human males.

Testicular cancer is the most common cancer in young men, and therefore it is of interest to screen all young men by the herein disclosed non-invasive method. Therefore it is an aspect of the present invention to screen all men, especially younger men for testicular cancer, i.e. CIS, by the detection of at least one, preferably two markers in semen and/or ejaculate samples from said men. Furthermore, it is of relevance to screen any male that has been diagnosed with testicular dysgenesis syndrome and/or any of the diseases and/or disorders related thereto. This includes males that have or have been diagnosed with low fertility/low sperm count, hypospadias, cryptorchidism and men who previously have been diagnosed with testicular cancer. The method herein disclosed may also be used as a means to follow the efficacy of a treatment for e.g. testicular cancer and to monitor the health state of an individual after said treatment has been discontinued, to spot relapse/reoccurrence of the cancer.

Samples to be analyzed for the presence of markers for CIS and related disorders may be prioritized to be conducted among risk groups, or it may be used as a screening method in men, preferably younger men i.e. before the normal age of onset of testicular cancer, including but not limited to the following groups:
Men (male human beings)
Younger men, i.e. before the normal age of onset of testicular cancer
Men in the reproductive age, wherein the reproductive age comprises the age group after puberty (sexual maturation).
Men with, by other means, proven testicular cancer before and after treatment
Men with a priori history of cryptorchidism or hypospadias.
Men showing suspicious microlithiasis patterns by ultrasound examination.
Men with fertility problems.

The younger men to be screened for the presence of CIS may be in the age-range of 5-50 years, such as 5-10 years, for example 10-15 years, such as 15-20 years, for example 20-25 years, such as 25-30 years, for example 30-35 years, such as 35-40 years, for example 40-45 years, such as 45-50 years. Preferably, the younger males are in the age-range of 5-40 years, such as 10 to 35 years, such as 15-35 years, such as 15-30 years, or such as 15-25 years.

Screening is of relevance for all males in the reproductive age, the reproductive age being the age after puberty and thus it is an aspect of the present invention to detect the presence of CIS cells in semen/ejaculate samples of males in the reproductive age.

An aspect of the present invention regards the use of the herein disclosed method on any semen or ejaculate sample that may be available. For example, men with infertility problems usually have a semen sample examined for testing the quality of the sperm; said sample may also be used for screening for CIS. Likewise, any expanded health investigation may include screening for testicular cancer; for instance this could be done on all males reporting for possible military service/draft.

Taking and Handling of the Sample

The sample to be examined may be collected by one or more of several methods. The primary method of sample collection will be collecting the ejaculate from masturbation or by mechanically stimulating the prostate or by other means mechanically stimulating an ejaculation. Another method is the collection of semen directly from the testis by expiration with a syringe. Alternatively the sample may be collected following sexual intercourse. The method of the present invention may also be performed on semen samples that have been preserved, such as frozen or otherwise preserved, prior to analysis.

Preferably the semen sample/ejaculate will be issued after a given abstinence period, wherein abstinence period is understood as the time period from the previous ejaculation. The abstinence period may be varied but a given range of days will be optimal. In one embodiment of the present invention, the abstinence period before collecting the ejaculate is 1-10 days, such as 1-2 days, for example 2-3 days, such as 3-4 days, for example 4-5 days, such as 5-6 days, for example 6-7 days, such as 7-8 days, for example 8-9 days, such as 9-10 days. In another embodiment of the present invention, the abstinence period before collecting the ejaculate is at least 1 day, such as 2 days, for example 3 days, such as 4 days, for example 5 days, such as 6 days, for example 7 days, such as 8 days, for example 9 days, such as 10 days.

In one embodiment of the invention, the abstinence period does not exceed 10 days, as this will reduce the quality of the sample.

In a further embodiment of the present invention, the release of neoplastic/CIS cells from the testis may be stimulated prior to collecting the ejaculate/semen sample. Stimulation of the testis may include ultrasound, massage of the testis or prostate, repeated ejaculations or drug agents such as, but not limited to, Colchicine or Viagra.

An average ejaculate volume for a male human being is about 2 ml to 5 ml. In one embodiment of the present invention, the whole ejaculate volume is used for analysis. This will increase the likelihood of obtaining a sample with cells expressing CIS markers. In another embodiment, a subset of the entire volume is used, in the range of 10-90%, such as 10-20%, for example 20-30%, such as 30-40%, for example 40-50%, such as 50-60%, for example 60-70%, such as 70-80%, for example 80-90%. Thus the volume used for the detection according to the present invention may be the default volume of the ejaculate sample or a standardized volume such as 5 ml, 4.75 ml, 4.5 ml, 4.25 ml, 4.0 ml, 3.75 ml, 3.5 ml, 3.25 ml, 3.0 ml, 2.75 ml, 2.5 ml, 2.25 ml, or 2.0 ml. The volumes may be adjusted by removal of part of the sample or by addition of an isotonic fluid as is known to a person skilled in the art. In a further embodiment, the spermatozoa are removed prior to the analysis leaving the seminal fluid and any CIS and other exfoliated cells behind in the sample to be analyzed. Likewise cells other than spermatozoa may be concentrated by various means i.e. gradient centrifugation or immunoprecipitation.

Sample Preparation

The sample may be collected in a labeled container or placed in a labeled container. The label of the container may contain a unique identification number and one part of the label may be transferable to a slide.

The sample is collected in a container which may contain stabilizing agents to aid in preservation of the sample and of the signal/marker quality in the sample. Stabilizing agents include but are not limited to pH-buffers, Protease-inhibitors, RNase inhibitors, fixatives and other compounds/components known to persons skilled in the art.

Collected samples may be stored at the site of collection at suitable temperature or they may be transported to local or external laboratories for preparation.

The sample may be processed in different ways in order to get optimal signal from any neoplastic/CIS cells present in the sample. Processing the sample may include, but is not limited to; filtration, precipitation, immunoprecipitation, flow-sorting, lyzing, centrifugation, cooling, freezing, heating or any other methods known to a person skilled in the art. Preferably, the sample is treated to allow optimal detection of potential CIS cells. This is for example performed by treating the sample in a manner that allows the cells of the sample to remain intact and as far as possible also retain their original morphology. Further, dependent on the method of analysis chosen, see below for details hereof, the sample is prepared to accommodate said analysis.

Analyzing the Sample

The sample may be analyzed for the presence of markers for the detection of CIS or related disorders using a variety of analyses. These analyses include methods for the detection of mRNA transcripts and/or proteins, including but not limited to Immunoassays, Immunostaining, Immunofluorescence, Immunohistochemistry (IHC), Direct IHC, Indirect IHC, Immunocytochemistry, In situ hybridization (ISH), Fluorescent ISH (FISH), FISH In Suspension (FISH-IS™), Western blot, Flow cytometry, FACS (fluorescence-activated cell sorting), ImageStream, Turtle Probes, target primed rolling circle PRINS, Luminex assay, PCR (polymerase chain reaction), qRT-PCR (quantitative reverse-transcriptase PCR or ‘real-time PCR’), Nested PCR, Mass spectrometry, ELISA (enzyme-linked immunosorbent assay; or enzyme immunoassay EIA), Indirect ELISA, Sandwich ELISA, Competitive ELISA, Rolling circle replication (or Rolling circle amplification), Radioimmunoassay (RIA), Magnetic immunoassay (MIA), Lateral flow tests (or Lateral Flow Immunochromatographic Assays), Turbidimetry, Complement fixation test, DNA microarray, Protein microarray, Northern blotting, Dot blot and Enzymatic activity. Thus it is an object of the present invention that any method of detecting any of the herein disclosed or referenced markers may be employed for the detection of CIS cells in a semen/ejaculate sample.

In one embodiment, the cells of the collected sample may be fixed onto a microscope slide by centrifugation or by other means (e.g. a smear). In one embodiment the cytospin technique (Shandon industries) is employed, and the sample is hereafter referred to as a semen cytospin. The semen cytospin is subsequently handled as a normal microscope slide.

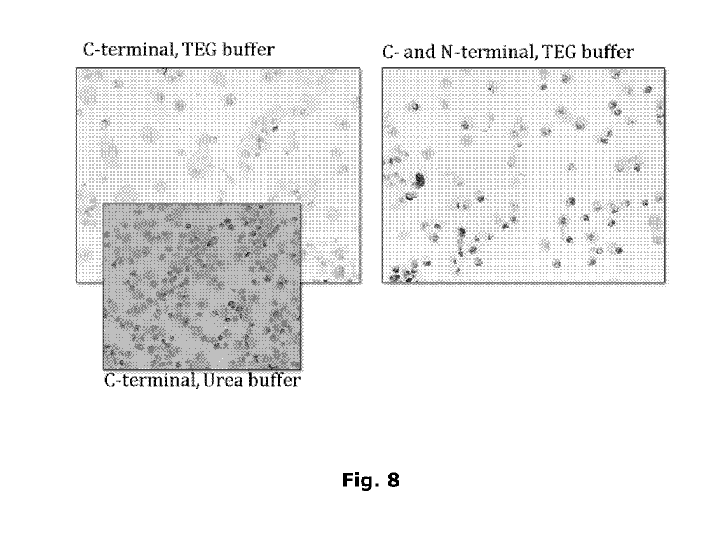

In a preferred embodiment, the analysis includes immunostaining of the sample for the detection of the at least two markers. The staining may be performed simultaneously in one-step, or may be performed in two or more subsequent staining procedures. The first step includes staining for a non-nuclear/cytoplasmic marker or a nuclear marker in the sample. The second step includes staining for the “reverse” marker i.e. staining for a nuclear marker if the first marker was a cytoplasmic/non-nuclear marker and vice versa. The sample may be a semen cytospin. The use of antibodies specific for any of the herein disclosed or referenced markers in a semen/ejaculate sample is in contrast with all previously published results wherein this method has proven non-feasible. Antibodies specific for a given marker may be used alone or in combinations. Combinations of antibodies are for example a combination of two or more antibodies directed against specific and most often different epitopes on the same marker. See for example FIG. 8, wherein staining with a combination of antibodies directed against the C- and N-terminus respectively of AP-2gamma gives an improved, more intense stain than the antibody against the C-terminus alone. When scoring the stains, a correlation between the stain type and the morphology of the stain/the cell is of importance: a nuclear stain in the shape of a nucleus gives a higher score than a nuclear stain on a morphology that is not immediately identifiable as nuclear. The same is given for the staining of a cytoplasmic protein or other morphologically specific markers, whether the stain is obtained by immunochemistry, enzymatic assay or other.

In a preferred embodiment a semen sample is collected and processed into a semen cytospin and subjected to analysis by immuno(cyto)staining using at least one, and preferably two or more antibodies each specific for any one of the abovementioned or referenced markers. Preferably, the markers are at least one of the markers listed in Table1, more preferably, the at least one marker is a marker listed in Table 2. Most preferably, two markers are sought detected by use of immunostaining, one marker being a marker with a nuclear localization the other with a non-nuclear localization. Further, it is preferred that any of the following nuclear markers are used for the detection of CIS in a cytospin or other prepared semen sample: TFAP2C, POU5F1, NANOG and TP53. Likewise, it is preferred that any of the following non-nuclear markers are used for the detection of CIS in a cytospin or other prepared semen sample: ALPPL2, ALPL, KIT and PDPN. It follows that it also is an object of the present invention that one or more antibodies specific for each marker and/or enzymatic assays for the same or spatially separate markers can be use simultaneously, separately and/or sequentially.

An object of the present invention regards the detection of cancerous cells in a semen/ejaculate sample by detection of at least one marker, such as but not limited to ALPP/ALPPL2, by use of an enzymatic assay. Thus, an embodiment regards the direct assay of ALPP/ALPPL2 by measuring the alkaline phosphatase activity of ALPP/ALPPL2. This assay includes, but is not limited to, the use of BCIP (5-Bromo-4-chloro-3-indolyl phosphate; a PLAP substrate) and NBT (nitro blue tetrazolium chloride; an oxidant), which is converted into a blue precipitate that can be visualized in a microscope. Optionally, kiel staining and levamisol (an inhibitor of endogenous phosphatases) may be used as well. Preferably, the enzymatic assay is performed on unfixed material such as a semen cytospin. This method may be used in combination with antibody staining for PLAP as well as any of the other markers herein disclosed.

In a preferred embodiment TFAP2C is detected by immunostaining and ALPP/ALPPL2 is detected by an enzymatic assay, preferably by the enzymatic assay of above.

The stained semen cytospin may be analyzed in different ways but in one preferred embodiment this is done by automated microscope scanning and subsequent automated image analysis. Alternatively, the results may be scored by manual microscopy, for instance by comparison to a scoring board indicating color depth/density/localization patterns or other parameters as are known to those skilled in the art.

In one embodiment, an automated slide scanner scans slides and the image stored on a computer system. The image is then analyzed by an algorithm designed to identify cancerous cells/CIS cells in the semen cytospin.

### DETAILED DESCRIPTION OF DRAWINGS

FIG. 1 The human male reproductive system. Schematic drawing of the male reproductive system showing the route cells from the testis during ejaculation. The major glands that may contribute cells to the ejaculate are the epididymis, the seminal vesicle, and the prostate.

FIG. 2 Testicular dysgenesis syndromes (TDS), its causes and outcomes. Schematic representation of pathogenic links between the components and clinical manifestations of testicular dysgenesis syndrome (adapted from Skakkebk et al. 2001). Adverse conditions in male reproductive health have their basis in a common origin; specific errors during the development of fetal testes. This collection of disorders is called testicular dysgenesis syndrome (TDS).

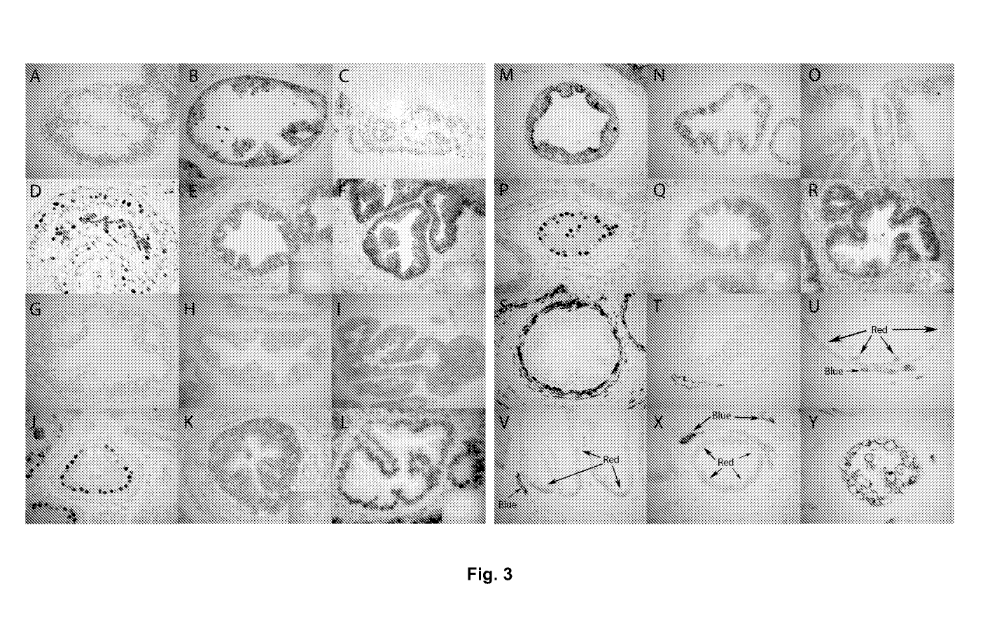

FIG. 3 Immunohistochemical and enzyme activity survey of expression of CIS markers and activity in epithelia from the urogenital tract: Immunohisto-chemical staining of selected markers in urogenital tissues. A-T: paraffin sections. U-Y: cryo sections. A-D: AP-2γ IHC. A: epididymis. B: prostate. C: seminal vesicle. D: testicular tubules (counter-stained with Meyer's haematoxyllin). E-F: AP-2γ ISH (inserts: sense control). E: epididymis. F: prostate. G-J: OCT3/4 IHC. G: epididymis. H: prostate. I: seminal vesicle. J: testicular tubules (counter-stained with Meyer's Haematoxyllin). K-L: OCT3/4 ISH (insert sense control). K: epididymis. L: prostate. M-P: NANOG IHC. M: epididymis. N: prostate. O: seminal vesicle. P: testicular tubule (counter-stained with Meyer's haematoxylin). Q-R: NANOG ISH (insert sense control). Q: epididymis. R: prostate. S: PLAP IHC epididymis. T: AGGRUS (podoplanin) IHC prostate. U: epididymis stained for alkaline phosphatase HC (blue) and AP-2γ IHC (red). V: prostate stained for alkaline phosphatase HC (blue) and OCT3/4 IHC (red). X: seminal vesicle stained for alkaline phosphatase HC (blue) and OCT3/4 IHC (red). Y: Testicular tubules with CIS stained for alkaline phosphatase HC (blue). HC: histochemistry, IHC: Immunohistochemistry, ISH: in situ hybridization. Results are outlined in table 3 and further explained in example 1.

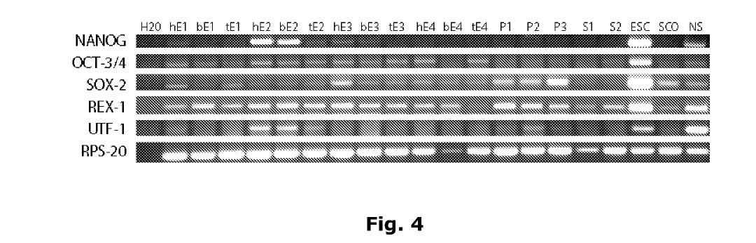

FIG. 4 RT-PCR of selected CIS markers in urogenital tissues. RT-PCR showing mRNA expression of pluripotency associated transcription factors in 4 different epididymis (E1-E4), 3 prostates (P1-P3), 2 seminal vesicles (S1 & S2), embryonic stem cells (ESC), Sertoli cell-only (SCO), and normal spermatogenesis (NS). Samples from epididymis are separated in head (hE), body (bE), and tail (tE).

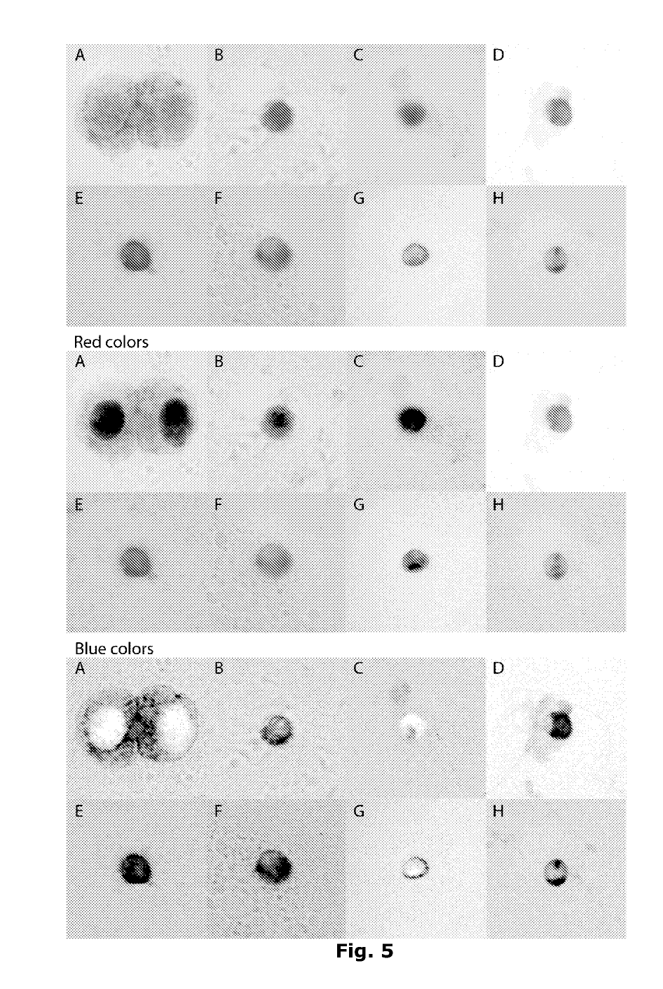

FIG. 5 Staining of CIS cells in semen samples using both alkaline phosphatase activity and AP-2γ immunohistochemistry. The top panel displays both blue and red colors while the middle shows red colors (always in the nucleus) and the panel at the bottom displays the blue colors (predominantly in the cytoplasm). Image A shows two cells with a blue stained cytoplasm and red nucleus that without doubt are CIS cells. However the spatial arrangement of a nucleus and a cytoplasm fixed on a slide can also be arranged with the cytoplasm on top of the nucleus as illustrated in FIGS. 5B, D, G and H. We also observed cells that were positive for either of the two markers used. FIG. 5C shows staining with only AP-2γ in FIGS. 5E and F cells positive only for alkaline phosphatase are shown.

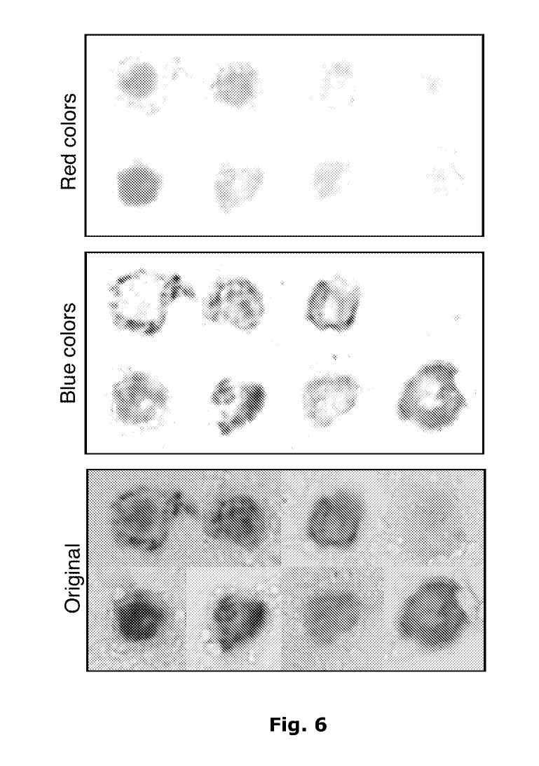

FIG. 6 Representative cells found in ejaculates from an infertile male for whom the diagnosis of CIS cells later was confirmed in a testicular biopsy. The figure is in black and white: the denotation “Red” is for the nuclear AP-2γ stain and “Blue” is for the alkaline phosphatase activity. “Original” is the overlay of the two stainings, “Red” and “Blue”.

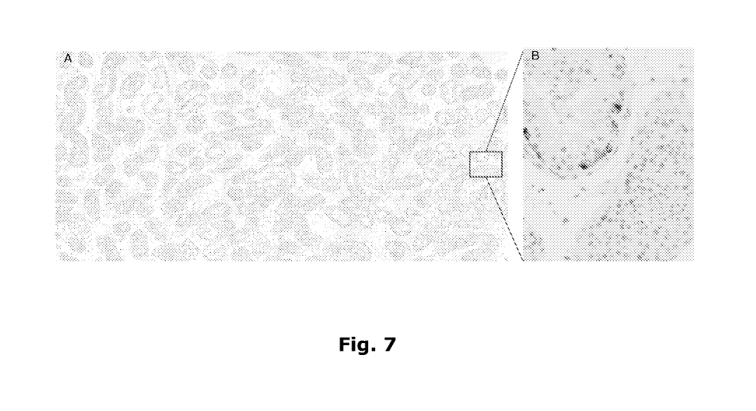

FIG. 7 Immunohistochemical staining for PLAP in a testicular biopsy taken from an infertile male with 4 out of 4 positive semen cytospins. The cytospins were positive in that CIS cells were found in each cytospin. A) Large overview. B) Higher magnification.

FIG. 8 Immunostaining of TCam-2 control cells demonstrating improved staining intensity when using a combination of antibodies directed towards different parts of the AP-2gamma protein (C- and N-termini) compared to antibodies against the C-terminal alone (as shown in the figure) or the N-terminus (not shown).

### EXAMPLES

### Example 1

To provide a method for performing a non-invasive test for CIS in human semen, we performed immunohistochemical analysis on epididymis, prostate and the seminal vesicle with antibodies against ALPP/ALPPL2, POU5F1, TFAP2C, NANOG, KIT and MAGE-A4.

In the below our findings are presented: a double staining for the cytochemical investigation for CIS in semen samples, based on histochemical (enzymatic) detection of placenta-like alkaline phosphatases (ALPP/ALPPL2) and immunocytochemical detection of Ap-2γ (TFAP2C).

Materials and Methods

Tissue Samples

The regional committee for Medical Research Ethics in Denmark approved the use of human tissue samples for this project. Samples included epididymis, prostate and vesicula seminalis from the archives of the Department of Pathology (Rigshospitalet) as well as malignant testicular material from the archives at University Department of Growth and Reproduction (Rigshospitalet). Additional and fresh material was obtained from the surplus of tissue after prostate and testis cancer surgery.

Immunohistochemistry (IHC)

Tissue samples from epididymis, prostate and vesicula seminalis were fixed overnight at 4° C. in buffered formalin or 4% paraformaldehyde PFA; positive control material in the same fixatives (CIS, seminoma or embryonal carcinoma) from the archives were used when needed. The following antibodies were used: OCT 3/4 (POU5F1) (monoclonal mouse antibody C-10; sc5279, Santa Cruz Biotechnology Inc., Santa Cruz, Calif.), NANOG (goat anti-NANOG AF1997; R&D Systems, Minneapolis, Minn., USA), C-kit (KIT) (monoclonal mouse anti-human C-kit CD117, Dako, Copenhagen), Ap-2γ (TFAP2C) (monoclonal mouse antibody 6E4/4; sc12762, Santa Cruz), PLAP (ALPPL2) (monoclonal mouse anti-human Placental Alkaline Phosphatase, clone 8A9, Dako) and MAGE-A4 (from G. Spagnoli, Ludwig Institute for Cancer Research, Lausanne, Switzerland). OCT3/4, NANOG, PLAP and Ap2γ are excellent markers for CIS whereas at least some CIS cells are positive for C-kit and MAGE-A4.

Immunohistochemical (IHC) protocols for the antibodies used on testis tissue have been described before; OCT3/4 (POU5F1) (Rajpert De-Meyts et al., 2004), NANOG (Hoei-Hansen et al, 2005b), C-Kit (KIT) (Rajpert-De Meyts et al., 2003a), Ap2γ (TFAP2C) (Hoei-Hansen et al, 2004), PLAP (ALPPL2) (Jacobsen and Norgaard-Pedersen, 1984) and MAGE-A4 (Aubry et al., 2001). In short, a standard indirect IHC protocol was used. Sections were de-waxed, rehydrated and de-masked by microwave treatment in 10 mM citrate buffer (pH 6.0) for NANOG, MAGE-A4 and C-Kit, in 5% urea (pH 8.5) for Ap2γ and OCT3/4 and in TEG buffer (pH 9.0; Tris 6.06 g/5 l and EGTA) for PLAP. Subsequently the sections were exposed to 0.5% H₂O₂ to inhibit endogenous peroxidase. Blockade of unspecific binding sites were performed with 2% non-immune goat serum (Zymed Histostain kit 95-6543, USA) for all antibodies except NANOG where human serum 1:4 in TBS was used. Antibodies were applied in the following dilutions; OCT3/4 (1:250), NANOG (1:20), Ap2γ (1:50), PLAP (1:100), MAGE-A4 (1:200) and C-Kit (1:400) and incubated overnight at 4° C. The concentrations of the used antibodies were the same as those normally used on malignant testicular material. Biotinylated goat anti-mouse IgG (Zymed Hitostain kit, USA) was used as a secondary antibody against the monoclonal antibodies, a biotinylated rabbit anti-goat antibody (Zymed kit 81-1640) was used against NANOG, and a peroxide-conjugated streptavidin complex was used as a tertiary layer. Visualization was performed with aminoethyl carbazole (Zymed Histostain kit).

In order to mimic the situation in semen samples as much as possible, the OCT3/4 and Ap2γ antibodies were used on cryo-sectioned material from epididymis and prostate as well. These cryo-experiments were conducted in combination with the BCIP/NBT reaction for Alkaline phosphatase as well (see below). Examination was done on a

Nikon Microphot-FXA microscope (Nikon, Japan) and results scored by two investigators. Staining was assessed using an arbitrary semi-quantitative score of staining intensity:

+++: strong staining

++ moderate staining

+: weak staining

+/− very weak staining

neg: no staining

Cytochemistry

Alkaline phosphatase activity (ALPP/ALPPL2) can be visualized directly on unfixed material with the substrate 5-bromo-4-indolyl phosphate/nitroblue tetrazolium (BCIP/NBT) (Sigma-Aldrich, USA). We used the protocol given in Nielsen et al. (2003) without levamisol; the inhibitor of endogenous phosphatases. BCIP/NBT staining for Alkaline phosphatase were performed on cryo-sections and on cytospin of human semen. For this purpose we used leftovers from infertile men, potential testis cancer patients and testis cancer patients, who signed an agreement for the use of surplus of their semen for scientific investigations. Semen was diluted to a concentration of approximately 25×10⁶ spermatozoa/ml in D-PBS (Gibco, Paisley, UK). Samples of 100 μl diluted semen followed by 400 μl PBS were applied to Shandon double cyto-funnels (5991039, Anatomical Pathology International Runcorn, UK) and centrifuged using a Shandon Cytospin 2 centrifuge at 1500 rpm for 5 min onto SuperFrost^{R}Plus microscope slides (Menzel-Glaser, Braunschwieg, Germany). The resultant slides were fixed in 75% ethanol, air dried and exposed to the developmental buffer for 10 seconds followed by BCIP/NBT substrate for 90 seconds and washed in running tab water. Finally the slides were fixed in 4% PFA in 10 min before exposure to IHC protocols.

RT-PCR

RT-PCR was performed using standard procedures as described elsewhere (Hoie-Hansen et al. 2005). Briefly, total RNA was purified using the NucleoSpin RNAII kit as described by the manufacturer (Macherey-Nagel, Düren, Germany) and samples were DNAse digested. cDNA was synthesized using a oligo dT and random hexamer primers and specific primers were designed for each gene. For control of PCR load and cDNA synthesis the expression of the marker gene RPS20 was analyzed. PCR products were run on 1.5% agarose gels and visualized by ethidium bromide staining.

In Situ Hybridization (ISH)

Probes for ISH were prepared by RT-PCR amplification by the use of specific primers spanning intron-exon boundaries. Probes were designed to detect NANOG1 and pseudo-genes, but not the described NANOG2 (Q8N7Ro, Ensemble.org) transcript. First primer combination CTGCTAAGGACAACATTGATAG and ATACAAGACCTCTTTCTACAAAG, second primer combination AATTAACCCTCACTAAAGGGCTTGCCTTGCTTT and TAATACGACTCACTATAGGGCGACACTATTCTC, the latter containing an added T3- or T7-promoter sequence, respectively (promoter sequences underlined). Likewise the primers for the AP-2γ probes: first primer combination AAGAGTTTGTTACCTACCTTACT and CATCAATTTGACATTTCAATGGC, second primer combination AATTAACCCTCACTAAAGGGTTAAAGAGCCTTCACT and TAATACGACTCACTATAGGGCTAAGTGTGTGG. Similarly for POU5F1 probes: first primer combination GGGTGGAGGAAGCTGACAAC and GCATAGTCGCTGCTTGATCG, second primer combination AATTAACCCTCACTAAAGGGCTGACAACAATGAAAAT and TAATACGACTCACTATAGGGGTTACAGAACCACACTC. PCR conditions included the following; 5 minutes at 95° C.; 5 cycles of 30 seconds at 95° C., 1 minute at 45° C., 1 minute at 72° C.; and 20 cycles of 30 seconds at 95° C., 1 minute at 65° C., 1 minute at 72° C. and finally 5 minutes at 72° C. The resulting PCR product was purified on a 2% low melting point agarose gel and sequenced from both ends, using Cy5-labelled primers complementary to the added T3 and T7 tags. Aliquots of ˜200 ng were used for in vitro transcription labeling, using the MEGAscript-T3 (sense) or MEGAscript-T7 (anti-sense) kits, as described by the manufacturer (Ambion/ABI, USA). To estimate quantity and labeling efficiencies, aliquots of the labeled RNA product were analyzed by agarose gel electrophoresis. ISH was performed as described previously (Nielsen et al., 2003). In brief, sections were re-fixed in 4% PFA, treated with proteinase K (Sigma-Aldrich, USA) (1.0-5.0 μg/ml), post-fixed in PFA, pre-hybridized 1 h at 50° C., and hybridized overnight at 50° C. with biotinylated antisense and sense control probes. Excess probe were removed with 0.1× standard saline citrate (60° C.). Visualization was performed using streptavidin conjugated with alkaline phosphatase (1:1000) (Roche Diagnostics, Germany) followed by a development with BCIP/NBT.

Results

AP-2γ (TFAP2C): Epididymis, prostate and seminal vesicle epithelium were positive for AP-2γ according to the IHC experiments and the results for epididymis and prostate were confirmed by ISH. In epididymis, the IHC staining for AP-2γ were strong to moderate and almost entirely nuclear. In prostate the IHC staining were intense to moderate, and the most intense and sometimes only staining was confirmed to the nuclei. In some instances, apparently almost liberated intensely stained nuclei were observed at the surface of the epithelium. In seminal vesicle epithelium the AP-2γ IHC detections were mainly in the nuclear membrane. This could be because of a generally weaker staining than in the two other kinds of epithelium investigated. IHC was performed on cryo-sectioned material as well and again the epithelium of epididymis and prostate were positive. The epididymis material used for cryo-experiments was divided in head, middle and tail. In the tail and middle part the detections were nuclear, whereas cytoplasmic to nuclear membrane staining was observed in the head part.

OCT3/4 (POU5F1): The immunohistochemical (IHC) staining for OCT3/4 were weak to very weak in epididymis, prostate and in the seminal vesicle. The IHC staining was restricted to the epithelium in all three glands. In epididymis, the staining was mainly nuclear (one sample was positive in the cytoplasm), and small round bodies in the nucleus frequently stained very intensively. In prostate and seminal vesicle, both nuclear and cytoplasmic staining occurred. These detections in epididymis and prostate could be confirmed with ISH.

NANOG: Epididymis, prostate and seminal vesicle epithelium were weakly positive for NANOG with IHC. In epididymis, the general staining pattern for NANOG was strong to moderate and most of the staining was localized to the nuclear membrane coupled with weaker staining in the cytoplasm of the epithelium, although a number of deviations from this were found. Such as scattered single epithelium cells with intense staining in the cytoplasm or scattered cells with intense staining of round bodies of nuclear size or much smaller one in each cell or in considerable amount per cell. In some tubules a strong staining towards the lumen was observed; this could be accounted for as an artifact do to better accessibility for antibodies at the edge of the cellular boundary. In one sample with embryonal carcinoma (intensively positive for NANOG) in epididymis, the tubule epithelium most proximate to the tumor stained considerably weaker than the tubule distant from the tumor. Faint staining of smooth muscle nuclei surrounding the tubules was observed here and there. The NANOG mRNA was detected by in situ hybridization in the epididymis epithelium. In prostate epithelium the IHC staining for NANOG was strong to moderate. NANOG was mainly found in the cytoplasm. Only a few nuclear detections were found and again scattered cells reacted more intensely. Seminal vesicle had positive to weak cytoplasmic IHC staining for NANOG.

C-Kit (KIT): The epithelia cell membranes of epididymis, prostate and seminal vesicle were negative using IHC staining with the KIT antibody. PLAP (ALPPL2) and MAGE-A4: The epithelium of epididymis, prostate and seminal vesicle were negative using PLAP and MAGE-A4 antibodies in IHC analysis, although the surrounding smooth muscle cells were strongly stained with the PLAP antibody. Staining of smooth muscle fibers with the Dako PLAP antibody is not unknown in routine investigations of malignant testis biopsies. This reaction is considered to be a background reaction caused by an unknown epitope in myoid cells, but some of the other PLAP antibodies have different restrictions. To verify the negative PLAP reactions, we used the BCIP/NBT reaction for Alkaline phosphatase on cryo-sectioned material from epididymis, prostate and seminal vesicle. The only positive reactions found were the endothelia cells in small blood vessels.

Epithelia Reactions

TABLE 3
Summarized results obtained with the used antibodies, histochemical reactions for Alkaline phosphatase
(BCIP/NBT) and ISH probes in epithelium of epididymis, prostate and seminal vesicle.

| | | | | | | | Vesicula | | |
|---|---|---|---|---|---|---|---|---|---|
| | IHC/ISH | n | Epididymis | n | Prostate | N | seminalis | CIS | NT |
| AP-2γ | IHC | 5 | ++ to +/− | 3 | +++ to + | 3 | + to +/− | +++ | neg |
| AP-2γ | IHC | 4 | ++ to +/− | 2 | +++ to +/− | 1 | + to +/− | +++ | neg |
| | Cryo | | | | | | | | |
| AP-2γ EV | IHC | 1 | ++ | 1 | +++ to ++ | 1 | + to +/− | +++ | neg |
| | Cryo | | | | | | | | |
| AP-2γ | ISH | 3 | +++ to +/− | 3 | +++ to + | 0 | n.d. | +++ | Spc, Spt +/− [symbol] |
| OCT 3/4 | IHC | 5 | + to +/− | 5 | + to +/− | 3 | + to +/− | +++ | neg |
| OCT 3/4 | IHC | 3 | + to +/− | 2 | + to +/− | 2 | + to +/− | +++ | neg |
| | Cryo | | | | | | | | |
| OCT 3/4 | ISH | 3 | +++ to + | 3 | +++ to + | 0 | n.d. | +++ | Spc, Spt +/− [symbol] |
| Nanog | IHC | 5 | +++ to + | 4 | ++ to + | 3 | + to +/− | +++ | neg |
| Nanog | ISH | 3 | +++ to + | 1 | ++ # | 0 | n.d. | +++ | GC +/− |
| C-Kit | IHC | 4 | neg | 3 | neg | 2 | neg | +++ | Spc ++ |
| C-Kit EV | IHC | 3 | Neg | 3 | neg | 2 | neg | +++ | Spc ++ |
| MAGE-A4 | IHC | 3 | Neg | 3 | neg | 2 | neg | +++/neg | Spg +++ |
| D2-40 | IHC | 2 | neg * | 3 | neg * | 2 | neg | +++ | neg |
| PLAP | IHC | 3 | Neg | 3 | neg | 2 | neg | +++ | neg |
| BCIP/NBT | HC | 3 | Neg | 3 | neg | 2 | neg | +++ | neg |

The two last columns (CIS; carcinoma in situ and NT; normal tubules) to the right summarize the results obtained with the same antibodies and the same protocols. IHC: Immunohistochemistry, ISH: In situ hybridization, HC: histochemistry. EV: Dako Envision system. Gc: germ cells. Spg: spermtogonia. Spc: spermatocytes. Spt: spermatids.
Staining was assessed using an arbitrary semi-quantitative score of staining intensity:
+++: strong staining,
++ moderate staining,
+: weak staining,
+/− very weak staining.
# some background staining observed but clearly a positive reaction. [symbol]
Mainly staining of nuclei. D2-40
* scattered epididymis, tubules and vesicles had weak cytoplasmic reaction in the basal epithelia cells.

RT-PCR

FIG. 4 shows RT-PCR of selected genes related to pluripotency. Pluripotency is a well-known hallmark of CIS cells and several of the embryonic characteristics' is even retained in overt seminomas. FIG. 4 shows that many of the genes responsible for the pluripotent phenotype also is expressed in the urogenital epithelia. The genes analyzed by RT-PCR are all transcription factors, which are located in the nucleus, and many of them also expressed in CIS cells. This further emphasizes that nuclear markers of CIS also is found expressed in the urogenital epithelia and thus could cause false positive staining in a semen cytospin analyzed for CIS cells.

Semen Cytology

A range of semen samples were analysed with the double staining procedure and scores given for each AP-2γ and alkaline phosphatase staining. Different combinations of positivity were observed as outlined in FIG. 5. Obviously a red nuclear staining with a blue staining as shown in FIG. 5A is without doubt a positive CIS cell. However the spatial arrangement of a nucleus and a cytoplasm fixed on a slide can also be with the cytoplasm on top of the nucleus as illustrated in FIGS. 5B, D, G and H. We also observed cells that were positive for either of the two markers used and shown with AP-2γ in FIG. 5C and alkaline phosphatase in FIGS. 5E and F. While a positive stain for AP-2γ indicates a high possibility for a true positive CIS cell this is not the case for the alkaline phosphatase as the reaction is somewhat unspecific as described earlier (Giwercman et al. 1990; Hoei-Hansen, C. E et al. 2007). However, positive staining of both AP-2γ and alkaline phosphatase in combination as in FIG. 5A or B significantly increases the likelihood of having a true positive CIS cell.

Discussion

This investigation shows that the classic good nuclear markers for CIS: AP-2γ (TFAP2C), OCT3/4 (POU5F1) and NANOG are also expressed in the epithelia of the urogenital system. Thus, false positive results in semen samples may well be due to liberated cells from these epithelia.

Our concern in this experiment has been to focus on the protocols for staining of CIS and compare these results with those obtained by this study on epididymis, prostate and seminal vesicle.

The nuclear staining of urogenital epithelia with OCT3/4, NANOG and AP-2γ could cause problems in a CIS semen test, due to the presence of exfoliated epithelial cells in the semen/ejaculate. HE staining of epididymis from testis cancer patients frequently show Eosine (red) positive globular bodies of various size within the nucleus and in the cytoplasm; similar globular bodies in the same microscopic observation field seems to be liberated into the lumen of the tubule. The Department of Pathology at Rigshospitalet experience that these hyalin globular structures might cause non-specific binding of antibodies. These structures could be the same we observed with intense staining with NANOG and OCT3/4. These globular bodies of nuclear size could be a problem in a CIS semen test, causing further false positive results. Apparently, AP-2γ positive nuclei are liberated from the prostate epithelium, and can thus be liberated into the seminal fluid.

The biological significance of these epithelia being positive for OCT3/4 and NANOG could be the need for continuous renewal of the epithelia and thereby a need for these genes mainly considered as purely stem cell genes. It is surprising that all the nuclei in the epithelium are positively stained. We have no suggestions as to the biology behind AP-2γ localization in these epithelia.

The negative results obtained for IHC staining with PLAP antibodies were confirmed with negative histochemical staining for alkaline phosphatase on cryo-sections in these epithelia. This surprisingly shows that in contrast to other well-characterized markers for CIS that also are expressed in the urogenital epithelium, PLAP (ALPPL2) is not expressed there. Thus we present the method of detection of cancerous cells in a semen/ejaculate sample by double staining of cancerous/CIS cells with BCIP/NBT for alkaline phosphatase and AP-2-γ immunostaining as is presented herein

### Example 2

Screening of infertile men for testicular cancer by double staining of semen samples. The purpose of this study was to follow the possibility of using the present immunocytological assay as a screening tool to identify patients with an asymptomatic CIS lesion among the risk group of subfertile males. In addition we have used semen samples from patients known to possess a testicular cancer to validate the performance of the assay in this group of patients.

Herein the cytological method is further improved and we have tested the performance in a cohort of 311 subfertile men referred to the hospital due to infertility. Four patients were identified as positive and at present one has later been diagnosed with CIS by surgical biopsy. 11 patients were identified as borderline and 292 negative.

Materials and Methods

Semen, Tissue and Cell Line Samples

The regional Committee for Medical Research Ethics in Denmark approved the use of human tissues for this project, including semen samples (permit nr. H-KF-012006-3472).

Semen Samples

The procedure was performed on fresh semen samples after routine semen analysis. If necessary, the samples were diluted to a concentration of 25×106 spermatozoa/ml in PBS (Invitrogen). Samples of 100 μl diluted semen were loaded into Shandon double cyto-funnels and 100 μl PBS were added before the samples were centrifuged using Shandon Cytospin 2 centrifuge (Anatomical Pathology International, Runcorn, UK) at 1500 rpm for 5 min onto SuperFrost Plus microscope slides (Menzel-Glaser, Braunschweig, Germany). Cytospins were prefixed in 75% ethanol and were first stained for alkaline phosphatase activity by submerging in a BCIP/NBT substrate for 5 min., followed by washing and post-fixation in 4% PFA for 10 min. A cytospin of TCam-2 cells was processed in parallel as a positive control. Cytospins were subsequently stored up to 4 days in refrigerator, before being stained for AP-2γ according to the previously published IHC protocol for semen samples, except that the contrast staining was omitted (Hoei-Hansen et al., 2007). Examination of the stained cytospins was done using a light microscope by at least two independent investigators. The TCam-2 cell line derived from a human seminoma (de Jong et al., 2008) was used to as a positive control for successful staining as these are positive for both TFAP2C and PLAP. TCam-2 cells were cultured using RPMI1640 supplemented with 10% fetal calf serum and penicillin/streptomycin (all from Invitrogen, San Diego, Calif., USA) at 37° C. in an incubator with 5% carbon dioxide, as previously described (Goddard et al.,2007).

Tissue Samples

Tissue samples were snap frozen or fixed overnight at 4° C. in buffered formalin or 4% paraformaldehyde (PFA). Immunohistochemistry on paraffin sections was performed using a standard indirect peroxidase method, using TFAP2C (monoclonal mouse antibody, MAb 6E4/4; sc-12762, Santa Cruz Biotechnology Inc., Santa Cruz, USA) as described in (Hoei-Hansen et al., 2005a), POU5F1 (MAb C-10; sc.5279, Santa Cruz) as described by (Rajpert-De Meyts et al., 2004) and placental-like alkaline phosphatase (PLAP) (MAb, clone 8A9, DAKO, recognises both ALPP and ALPPL2), as described in (Givercman et al., 1991; Jacobsen and Noergaard-Pedersen, 1984).

Patients

Semen samples were obtained from 311 patients attending our andrology clinic for semen analysis and/or cryopreservation. The patients were enrolled in the study after written informed consent, and if at least 100 μl (microliter) of the sample was available after standard semen analysis and cryopreservation.

Two different categories of patients were analyzed: Patients attending the fertility clinic for infertility reasons and thus only came to the hospital in order to become a father and patients under suspicion of having a testicular cancer.

Results

Screening of Subfertile Males

In an 8 months period we have analyzed 439 cytospins from 311 subfertile patients. In most cases 1 or 2 ejaculates per patient were analyzed but in some cases up to 4 samples from the same patient were analyzed (4 samples n=2; 3 samples n=2; 2 samples n=119; 1 sample n=188). Only a small fraction (100-400 ul) of the ejaculate was analyzed after routine semen analysis. The mean sperm concentration was 19.4*10E6 sperm/ml and the mean age 34.2.

292 males were scored as negative (grade 0-2) equalling 93.9% of the males and 11 males (3.5%) were deemed borderline (grade 3). 4 males were found positive (grade 4-5) equalling 1.3% of the subfertile males investigated. Results are outline in Table 4. Two of the positive males delivered 4 independent ejaculates each and all 4 samples were found positive in both cases. Representative cells are shown in FIG. 1. A testicular biopsy was taken for one of the males with 4 out of 4 positive samples and diagnosed with CIS (FIG. 2). The other positive male is still in clinical follow-up and no definitive clinical conclusion is reached yet.

Among the borderline cases one was biopsied and no CIS could be identified.

TABLE 4
Summarized results of experiments conducted
as described in Example 2.

| | Patients | % of total | Cytospins | % of total |
|---|---|---|---|---|
| Total subfertile* | 311 | 100 | 439 | 100 |
| Negative | 292 | 93.9 | 403 | 91.8 |
| Borderline | 11 | 3.5 | 19 | 4.3 |
| Positive | 4 | 1.3 | 10 | 2.3 |
| Technical problem | 4 | 1.3 | 7 | 1.6 |
| Discrepancy (samples from | 4 | 1.3 | | |
| same patient) | | | | |

*Average semen concentration 19.4 * 10E6 per ml
Average age 34.2

Discussion

Within the group of infertile males different numbers of testicular cancer cases have been reported. In a large prospective study of 22562 males evaluated for infertility in the US the incidence ratio of testicular cancer cases was found to be 1.3. Limiting the evaluation to male infertility increased the incidence ratio to 2.8 (Walsh et al., 2009). Somewhat similar incidence ratios of 1.6 and 2.3 respectively, was also found among 32 442 Danish infertile men (Jacobsen et al., 2000). These numbers fit very well with the incidence of CIS positive males indentified in this study, where (4 out of 311) 1.3% was identified as positive. Among the 292 men with negative findings there might be someone who nevertheless develops a testicular cancer and possibly a couple of the borderline cases also harbour CIS, thus the incidence ratio may increase. In an earlier study from our group (Hoei-Hansen et al., 2007) a range of false positive infertile males were identified and the assay, which only included immunocytochemical staining for AP-2γ (TFAP2C), was used deemed only useful as an auxiliary technique to other established techniques (primarily ultrasound) and indicative risk factors (previous history of cryptorchidism or testicular dysgenesis). In the present study we have taken advantage of recent advances in the assay of CIS identification by means of double staining. Hence, immunocytochemical staining for AP-2γ was preceded by additional cytochemical staining for alkaline phosphatase enzymatic activity. This resulted in additional blue staining in the cytoplasm of CIS cells as illustrated in FIG. 1 and made grading decisions easier as positive double staining in combination with correct morphology definitively lead to a positive judgment.

### Example 3

Antibody combinations. The combination of antibodies directed against the N- and C-terminal of the AP-2γ protein, gives better intensity of the AP-2γ staining than with the C- or N-terminal alone. FIG. 8 shows staining in parallel-processed cytospins with TCam-2 control cells. Note the darker stains in the cytospin stained with the combination of antibodies compared to the C-terminal antibody alone.

### REFERENCE LIST

Almstrup K, Leffers H, Lothe R A, Skakkebaek N E, Sonne S B, Nielsen J E, Rajpert-De Meyts E, Skotheim R I. Improved gene expression signature of testicular carcinoma in situ. Int J Androl. 2007 August; 30(4):292-302; discussion 303.

Aubry, F., Satie, A. P., Rioux-Leclercq, N. Rajpert-De Meyts, E., Spagnoli, G. C., Chomez, P., De Backer, O., Jôgou, B. and Samson, M. (2001) MAGE-A4, a germ cell specific marker, is expressed differentially in testicular tumors. Cancer 92,2778-85.

Czaplicki, M. Rojewska, J., Pykalo, R. and Szymanska, K. (1987) Detection of testicular neoplasms by cytological examination of seminal fluid. J Urol 138,787-788.

de Jong J, Stoop H, Gillis A J, Hersmus R, van Gurp R J, van de Geijn G J, van Drunen E, Beverloo H B, Schneider D T, Sherlock J K, Baeten J, Kitazawa S, van Zoelen E J, van Roozendaal K, Oosterhuis J W, Looijenga L H. Further characterization of the first seminoma cell line TCam-2. Genes Chromosomes Cancer. 2008 March; 47(3):185-96

Giwercman, A., Marks, A., Bailey, D., Baumal, R. and Skakkebaek, N. E. (1988) A monoclonal antibody as a marker for carcinoma in situ germ cells of the human adult testis. ACta Pathol Micobiol Immunol Scand 96,667-670.

Giwercman, A., Hopman, A. H., Ramaekers, F. C. and Skakkebaek, N. E. (1990) Carsinoma in situ of the testis. Detection of malignant germ cells in seminal fluid by means of in situ hybridization. Am J Pathol 136,497-502.

Giwercman A, Cantell L, Marks A. Placental-like alkaline phosphatase as a marker of carcinoma-in-situ of the testis. Comparison with monoclonal antibodies M2A and 43-9F. APMIS 1991; 99:586-94.

Goddard N, McIntyre A, Summersgill B, Gilbert D, Kitazawa S, Shipley J. KIT and RAS signalling pathways in testicular germ cell tumours: new data and a review of the literature. Int J Androl 2007; 30:337-48; discussion 49.

Hoei-Hansen, C. E., Nielsen, J. E., Almstrup, K., Brask, S. B., Graem, N., Skakkebaek, N. E., Leffers, H., and Rajpert-de Meyts, E. (2004). Transcription factor AP-2γ is a developmentally regulated marker of testicular carsinoma in situ and germ cell tumors. Clin. Cancer Res. 10, 8521-30

Hoei-Hansen, C. E., Rajpert-De Meyts, E., Carlsen, E., Almstrup, K., Leffers, H. and Skakkebaek, N. E. (2005a) A subfertile patient diagnosed with testicular carcinoma in situ by immunocytological staining for AP-2gamma in semen samples: case report. Hum Reprod 20,579-582.

Hoei-Hansen, C. E., Almstrup, K., Nielsen, J. E., Brask, S. S., Graem, N., Skakkebaek, N. E., Leffers, H., and Rajpert-de Meyts, E. (2005b). Stem cell pluripotency factor NANOG is expressed in human fetal gonocytes, testicular carcinoma in situ and germ cell tumours. Histopathology. 47, 48-56.

Hoei-Hansen, C. E., Carlsen, E., Jorgensen, N., Leffers, H. Skakkebaek, N. E. and Rajpert-De Meyts, E. (2007) Towards a non-invasive method for early detection of testicular neoplasia in semen samples by identification of fetal germ cell-specific markers. Hum Reprod 22,167-173.

Jacobsen, G. K. and Norgaard-Pedersen, B. (1984). Placental alkaline phosphatase in testicular germ cell tumours and in carcinoma-in-situ of the testis. An immunohistochemical study. Acta Pathol. Microbiol. Immunol. Scand. [A]. 92, 323-329.

Jacobsen R, Bostofte E, Engholm G, et al. Risk of testicular cancer in men with abnormal semen characteristics: cohort study. BMJ 2000; 321:789-92.

Meng, F. J., Zhou, Y., Skakkebaek, N. E., Marks, A. and Giwercman, A. (1996) Detection and enrichment of carcinoma-in-situ cells in semen by an immunomagnetic method using monoclonal antibody M2A. Int J androl 19,365-370.

Meng, F Zhou, Y., Giwercman, A. Skakkebaek, N. E., Geurts van Kessel, A. D. and Suijkerbuijk, R. F. (1998) Fluorescence in situ hybridization analysis of chromosome 12 anomalies in semen cells from patients with carcinoma in situ of the testis. j Pathol 186,235-239.

Nielsen, J. E., Hansen, M. A., Jorgensen, M., Tanaka, M., Almstrup, K., Skakkebaek, N. E., and Leffers, H. (2003). Germ cell differentiation-dependent and stage-specific expression of LANCL1 in rodent testis. Eur. J. Histochem. 47, 215-222.

Rajpert-De Meyts E. Developmental model for the pathogenesis of testicular carcinoma in situ: genetic and environmental aspects. Hum Reprod Update. 2006 12(3):303-23.

Rajpert-de Meyts, E., Jacobsen, G. K., Bartkova, J., Aubry, F., Samson, M., Bartek, J., and Skakkebaek, N. E. (2003a). The immunohistochemical expression pattern of Chk2, p53, p19INK4d, MAGE-A4 and other selected antigens provides new evidence for the premeiotic origin of spermatocytic seminoma. Histopathology. 42, 217-226.

Rajpert-De Meyts, E., Bartkova, J., Samson, M., Hoei-Hansen, C. E., Frydenlund-Larsen, L., Bartek, J. and Skakkebaek, N. E. (2003b) The emerging phenotype of the testicular carcinoma in situ germ cell. APMIS 111, 267-279.

Rajpert-de Meyts, E., Hanstein, R., Jorgensen, N., Graem, N., Vogt, P. H., and Skakkebaek, N. E. (2004). Developmental expression of POU5F1 (OCT-3/4) in normal and dysgenetic human gonads. Hum. Reprod. 19, 1338-1344.

Roth, M., Rajpert-de Meyts, E., Anderson, L., Dieckmann, K: P., Fossa, S. D., Grigor, K. M., Hendry, W. F., Herr, H. W., Looijenga, L. H., Oosterhuis, J. W. and Skakkebaek, N. E. (2000) Carsinoma in situ testis. Scand J Urol Nephrol Suppl 20S, 166-186.

Skakkebaek, N. E. (1972) Possible carsinoma-in-situ of the testis. Lancet 2, 516-517.

Skakkebaek N E, Rajpert-De Meyts E, Main K M. (2001) Testicular dysgenesis syndrome: an increasingly common developmental disorder with environmental aspects. Hum Reprod. 16(5):972-8.

Spreger et al; “Gene expression patterns in human embryonic stem cells and human pluripotent germ cell tumors”, (PNAS, 100:13350-13355, 2003)

Walsh T, Croughan M, Schembri M, Chan J, Turek P. Increased risk of testicular germ cell cancer among infertile men. Arch Intern Med 2009; 169:351-6.

1. A method for the detection of testicular cancer and/or precursors hereof by screening a sample for the presence of at least two markers in the same cell, wherein the sample is a semen sample and/or an ejaculate from a male human being.

2. The method according to claim 1, wherein of the at least two markers, at least one marker is a nuclear marker, and at least one other marker is a non-nuclear marker.

3. The method according to claim 1, wherein of the at least two markers, at least two are nuclear markers.

4. The method according to claim 1, wherein of the at least two markers, at least two are cytoplasmic markers.

5. The method according to any of claims 1 to 3, wherein the at least one nuclear marker is selected from the group consisting of: TFAP2C (Ap-2gamma), POU5F1 (OCT3/4), NANOG, SOX2, SOX15, SOX17, E2F1, IFI16, TEAD4, TLE1, TATDN2, NFIB, LMO2, MECP2, HHEX, XBP1, RRS1, MYCN, ETV4, ETV5, MYCL1, HIST1H1C, WDHD1, RCC2, TP53, and MDC1.

6. The method according to any of claims 1 to 3, wherein the at least one nuclear marker is selected from the group consisting of: TFAP2C (Ap-2gamma), POU5F1, NANOG and TP53.

7. The method according to any of claim 1 to 2, or 4, wherein the cytoplasmic marker is selected from the group consisting of ALPPL2 (PLAP), ALPL, DPPA4, TCL1A, CDH1, GLDC, TCL1A, DPPA4, CDK5, CD14, FGD1, NEURL, HLA-DOA, DYSF, MTHFD1, ENAH, ZDHHC9, NME1, SDCBP, SLC25A16, ATP6AP2, PODXL, PDK4, PCDH8, RAB15, EVI2B, LRP4, B4GALT4, CHST2, FCGR3A, CD53, CD38, PIGL, CKMT1B, RAB3B, NRCAM, KIT, ALK2, PDPN, HRASLS3, and TRA-1-60.

8. The method according to any of claim 1 to 2, or 4, wherein the cytoplasmic marker is selected from the group consisting of ALPPL2 (PLAP), ALPL, KIT and PDPN.

9. The method according to any of the preceding claims, wherein the at least two markers are Ap-2γ (TFAP2C) and PLAP (ALPPL2).

10. The method according to any of the preceding claims, wherein the examination of the semen sample and/or ejaculate comprises at least one of the following methods: Immunoassay, Immunostaining, Immunofluorescence, Immunohistochemistry (IHC), Direct IHC, Indirect IHC, Immunocytochemistry, In situ hybridization (ISH), Fluorescent ISH (FISH), FISH In Suspension (FISH-IS™), Western blot, Flow cytometry, FACS (fluorescence-activated cell sorting), ImageStream, Turtle Probes, target primed rolling circle PRINS, Luminex assay, PCR (polymerase chain reaction), qRT-PCR (quantitative reverse-transcriptase PCR or ‘real-time PCR’), Nested PCR, Mass spectrometry, ELISA (enzyme-linked immunosorbent assay; or enzyme immunoassay EIA), Indirect ELISA, Sandwich ELISA, Competitive ELISA, Rolling circle replication (or Rolling circle amplification), Radioimmunoassay (RIA), Magnetic immunoassay (MIA), Lateral flow tests (or Lateral Flow Immunochromatographic Assays), Turbidimetry, Complement fixation test, DNA microarray, Protein microarray, Northern blotting, Dot blot and/or Enzymatic activity.

11. The method according to any of the preceding claims, wherein the examination of the semen/ejaculate comprises immunostaining and/or an enzymatic assay.

12. The method according to any of the preceding claims, wherein the enzymatic assay comprises detection of alkaline phosphatase activity.

13. The method according to any of the preceding claims, wherein TFAP2C (Ap-2gamma) is detected by immunostaining and PLAP is detected by the method of claim 12,

14. The method according to any of the preceding claims, wherein testicular cancer comprises at least one of the following: testicular carcinoma in situ (CIS), germ cell tumor (TGCT), non-germ cell tumors of the testis or secondary tumor of the testis.

15. The method according to any of the preceding claims, wherein the testicular cancer is a carcinoma in situ (CIS).

16. The method according to any of the preceding claims, wherein the testicular germ cell tumor is a mixed tumor, a seminoma, an embryonal carcinoma, a teratoma, a choriocarcinoma or intratubular germ cell neoplasms (CIS).

17. The method according to any of the preceding claims, wherein the whole volume of the ejaculate is used for examination.

18. The method according to any of the preceding claims, wherein a subset of the entire volume is used, in the range of 10-90%, such as 10-20%, for example 20-30%, such as 30-40%, for example 40-50%, such as 50-60%, for example 60-70%, such as 70-80%, for example 80-90%.

19. The method according to any of the preceding claims, wherein the ejaculate is collected after at least 1 day of abstinence, such as 2 days, for example 3 days, such as 4 days, for example 5 days, such as 6 days, for example 7 days, such as 8 days, for example 9 days, such as 10 days of abstinence.

20. The method according to any of the preceding claims, wherein the ejaculate sample is taken from a younger male human being, wherein the younger male is in the age-range of 5-50 years, such as 10 to 35 years, such as 15-35 years, such as 15-30 years, or such as 15-25 years.

21. The method according to any of the preceding claims, wherein the ejaculate sample is taken from a male with, by other means, proven testicular cancer before and after treatment; a male with a priori history of cryptorchidism; a male showing suspicious microlits patterns by ultrasound examination; or a male with fertility problems.

22. The method according to any of the preceding claims, wherein said method comprises fixing of the semen/ejaculate onto a microscope slide.

23. The method according to any of the preceding claims, wherein the ejaculate is fixed by means of a cytospin.

24. Use of the method according to any of claims 1 to 23 for screening for the presence of testicular cancer in a semen/ejaculate sample of a male human being.
